SEQUENCE LISTING (9 sequences)

SEQ ID NO: 1 - protein, 22 aa, Porcine teschovirus-1
GSGATNFSLLKQAGDVEENPGP

SEQ ID NO: 2 - protein, 1305 aa, synthetic construct
MDAMKRGLCCVLLLCGAVFVSPSQEIHARFRRGARSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARGGGGDTGLSKNPIELVEGWFSSWKSSIASFFFIIGLIIGLFLVLRVGIHLCIKLKHTKKRQIYTDIEMNRLGKIDTVDGSGATNFSLLKQAGDVEENPGPMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAAYKLFDRYFKYWDQTYHPNCVNCLDDRCILHCANFNVLFSTVFPPTSFGPLVRKIFVDGVPFVVSTGYHFREDYKDDDDK

SEQ ID NO: 3 - protein, 18 aa, Thosea asigna virus
EGRGSLLTCGDVEENPGP

SEQ ID NO: 4 - protein, 19 aa, Porcine teschovirus-1
ATNFSLLKQAGDVEENPGP

SEQ ID NO: 5 - protein, 20 aa, Equine rhinitis A virus
QCTNYALLKLAGDVESNPGP

SEQ ID NO: 6 - protein, 22 aa, Foot-and-mouth disease virus 18
VKQTLNFDLLKLAGDVESNPGP

SEQ ID NO: 7 - protein, 33436 aa, synthetic construct
GCATCATCAATAATATACCTCAAACTTTTTGTGCGCGTTAATATGCAAATGAGGCGTTTGAATTTGGGGAGGAAGGGCGGTGATTGGTCGAGGGATGAGCGACCGTTAGGGGCGGGGCGAGTGACGTTTTGATGACGTGGTTGCGAGGAGGAGCCAGTTTGCAAGTTCTCGTGGGAAAAGTGACGTCAAACGAGGTGTGGTTTGAACACGGAAATACTCAATTTTCCCGCGCTCTCTGACAGGAAATGAGGTGTTTCTGGGCGGATGCAAGTGAAAACGGGCCATTTTCGCGCGAAAACTGAATGAGGAAGTGAAAATCTGAGTAATTTCGCGTTTATGGCAGGGAGGAGTATTTGCCGAGGGCCGAGTAGACTTTGACCGATTACGTGGGGGTTTCGATTACCGTGTTTTTCACCTAAATTTCCGCGTACGGTGTCAAAGTCCGGTGTTTTTACGTAGTTGACATTGATTATTGACTAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGTACATGACCTTATGGGACTTTCCTACTTGGCAGTACATCTACGTATTAGTCATCGCTATTACCATGGTGATGCGGTTTTGGCAGTACATCAATGGGCGTGGATAGCGGTTTGACTCACGGGGATTTCCAAGTCTCCACCCCATTGACGTCAATGGGAGTTTGTTTTGGCACCAAAATCAACGGGACTTTCCAAAATGTCGTAACAACTCCGCCCCATTGACGCAAATGGGCGGTAGGCGTGTACGGTGGGAGGTCTATATAAGCAGAGCTCTTAATTAATAATGAATTCATGCCACCATATCGATTATATCTAGATATAAGACATGATAAGATACATTGATGAGTTTGGACAAACCACAACAAGAATGCAGTGAAAAAAATGCTTTATTTGTGAAATTTGTGATGCTATTGCTTTATTTGTAACCATTATAAGCTGCAATAAACAAGTTAACAACAACAATTGCATTCATTTTATGTTTCAGGTTCAGGGGGAGATGTGGGAGGTTTTTTAAAGCAAGTAAAACCTCTACAAATGTGGTACATATGCTGGCCACCGTACATGTGGCTTCCCATGCTCGCAAGCCCTGGCCCGAGTTCGAGCACAATGTCATGACCAGGTGCAATATGCATCTGGGGTCCCGCCGAGGCATGTTCATGCCCTACCAGTGCAACCTGAATTATGTGAAGGTGCTGCTGGAGCCCGATGCCATGTCCAGAGTGAGCCTGACGGGGGTGTTTGACATGAATGTGGAGGTGTGGAAGATTCTGAGATATGATGAATCCAAGACCAGGTGCCGAGCCTGCGAGTGCGGAGGGAAGCATGCCAGGTTCCAGCCCGTGTGTGTGGATGTGACGGAGGACCTGCGACCCGATCATTTGGTGTTGCCCTGCACCGGGACGGAGTTCGGTTCCAGCGGGGAAGAATCTGACTAGAGTGAGTAGTGTTCTGGGGCGGGGGAGGACCTGCATGAGGGCCAGAATAACTGAAATCTGTGCTTTTCTGTGTGTTGCAGCAGCATGAGCGGAAGCGGCTCCTTTGAGGGAGGGGTATTCAGCCCTTATCTGACGGGGCGTCTCCCCTCCTGGGCGGGAGTGCGTCAGAATGTGATGGGATCCACGGTGGACGGCCGGCCCGTGCAGCCCGCGAACTCTTCAACCCTGACCTATGCAACCCTGAGCTCTTCGTCGTTGGACGCAGCTGCCGCCGCAGCTGCTGCATCTGCCGCCAGCGCCGTGCGCGGAATGGCCATGGGCGCCGGCTACTACGGCACTCTGGTGGCCAACTCGAGTTCCACCAATAATCCCGCCAGCCTGAACGAGGAGAAGCTGTTGCTGCTGATGGCCCAGCTCGAGGCCTTGACCCAGCGCCTGGGCGAGCTGACCCAGCAGGTGGCTCAGCTGCAGGAGCAGACGCGGGCCGCGGTTGCCACGGTGAAATCCAAATAAAAAATGAATCAATAAATAAACGGAGACGGTTGTTGATTTTAACACAGAGTCTGAATCTTTATTTGATTTTTCGCGCGCGGTAGGCCCTGGACCACCGGTCTCGATCATTGAGCACCCGGTGGATCTTTTCCAGGACCCGGTAGAGGTGGGCTTGGATGTTGAGGTACATGGGCATGAGCCCGTCCCGGGGGTGGAGGTAGCTCCATTGCAGGGCCTCGTGCTCGGGGGTGGTGTTGTAAATCACCCAGTCATAGCAGGGGCGCAGGGCATGGTGTTGCACAATATCTTTGAGGAGGAGACTGATGGCCACGGGCAGCCCTTTGGTGTAGGTGTTTACAAATCTGTTGAGCTGGGAGGGATGCATGCGGGGGGAGATGAGGTGCATCTTGGCCTGGATCTTGAGATTGGCGATGTTACCGCCCAGATCCCGCCTGGGGTTCATGTTGTGCAGGACCACCAGCACGGTGTATCCGGTGCACTTGGGGAATTTATCATGCAACTTGGAAGGGAAGGCGTGAAAGAATTTGGCGACGCCTTTGTGCCCGCCCAGGTTTTCCATGCACTCATCCATGATGATGGCGATGGGCCCGTGGGCGGCGGCCTGGGCAAAGACGTTTCGGGGGTCGGACACATCATAGTTGTGGTCCTGGGTGAGGTCATCATAGGCCATTTTAATGAATTTGGGGCGGAGGGTGCCGGACTGGGGGACAAAGGTACCCTCGATCCCGGGGGCGTAGTTCCCCTCACAGATCTGCATCTCCCAGGCTTTGAGCTCGGAGGGGGGGATCATGTCCACCTGCGGGGCGATAAAGAACACGGTTTCCGGGGCGGGGGAGATGAGCTGGGCCGAAAGCAAGTTCCGGAGCAGCTGGGACTTGCCGCAGCCGGTGGGGCCGTAGATGACCCCGATGACCGGCTGCAGGTGGTAGTTGAGGGAGAGACAGCTGCCGTCCTCCCGGAGGAGGGGGGCCACCTCGTTCATCATCTCGCGCACGTGCATGTTCTCGCGCACCAGTTCCGCCAGGAGGCGCTCTCCCCCCAGGGATAGGAGCTCCTGGAGCGAGGCGAAGTTTTTCAGCGGCTTGAGTCCGTCGGCCATGGGCATTTTGGAGAGGGTTTGTTGCAAGAGTTCCAGGCGGTCCCAGAGCTCGGTGATGTGCTCTACGGCATCTCGATCCAGCAGACCTCCTCGTTTCGCGGGTTGGGACGGCTGCGGGAGTAGGGCACCAGACGATGGGCGTCCAGCGCAGCCAGGGTCCGGTCCTTCCAGGGTCGCAGCGTCCGCGTCAGGGTGGTCTCCGTCACGGTGAAGGGGTGCGCGCCGGGCTGGGCGCTTGCGAGGGTGCGCTTCAGGCTCATCCGGCTGGTCGAAAACCGCTCCCGATCGGCGCCCTGCGCGTCGGCCAGGTAGCAATTGACCATGAGTTCGTAGTTGAGCGCCTCGGCCGCGTGGCCTTTGGCGCGGAGCTTACCTTTGGAAGTCTGCCCGCAGGCGGGACAGAGGAGGGACTTGAGGGCGTAGAGCTTGGGGGCGAGGAAGACGGACTCGGGGGCGTAGGCGTCCGCGCCGCAGTGGGCGCAGACGGTCTCGCACTCCACGAGCCAGGTGAGGTCGGGCTGGTCGGGGTCAAAAACCAGTTTCCCGCCGTTCTTTTTGATGCGTTTCTTACCTTTGGTCTCCATGAGCTCGTGTCCCCGCTGGGTGACAAAGAGGCTGTCCGTGTCCCCGTAGACCGACTTTATGGGCCGGTCCTCGAGCGGTGTGCCGCGGTCCTCCTCGTAGAGGAACCCCGCCCACTCCGAGACGAAAGCCCGGGTCCAGGCCAGCACGAAGGAGGCCACGTGGGACGGGTAGCGGTCGTTGTCCACCAGCGGGTCCACCTTTTCCAGGGTATGCAAACACATGTCCCCCTCGTCCACATCCAGGAAGGTGATTGGCTTGTAAGTGTAGGCCACGTGACCGGGGGTCCCGGCCGGGGGGGTATAAAAGGGTGCGGGTCCCTGCTCGTCCTCACTGTCTTCCGGATCGCTGTCCAGGAGCGCCAGCTGTTGGGGTAGGTATTCCCTCTCGAAGGCGGGCATGACCTCGGCACTCAGGTTGTCAGTTTCTAGAAACGAGGAGGATTTGATATTGACGGTGCCGGCGGAGATGCCTTTCAAGAGCCCCTCGTCCATCTGGTCAGAAAAGACGATCTTTTTGTTGTCGAGCTTGGTGGCGAAGGAGCCGTAGAGGGCGTTGGAGAGGAGCTTGGCGATGGAGCGCATGGTCTGGTTTTTTTCCTTGTCGGCGCGCTCCTTGGCGGCGATGTTGAGCTGCACGTACTCGCGCGCCACGCACTTCCATTCGGGGAAGACGGTGGTCAGCTCGTCGGGCACGATTCTGACCTGCCAGCCCCGATTATGCAGGGTGATGAGGTCCACACTGGTGGCCACCTCGCCGCGCAGGGGCTCATTAGTCCAGCAGAGGCGTCCGCCCTTGCGCGAGCAGAAGGGGGGCAGGGGGTCCAGCATGACCTCGTCGGGGGGGTCGGCATCGATGGTGAAGATGCCGGGCAGGAGGTCGGGGTCAAAGTAGCTGATGGAAGTGGCCAGATCGTCCAGGGCAGCTTGCCATTCGCGCACGGCCAGCGCGCGCTCGTAGGGACTGAGGGGCGTGCCCCAGGGCATGGGATGGGTAAGCGCGGAGGCGTACATGCCGCAGATGTCGTAGACGTAGAGGGGCTCCTCGAGGATGCCGATGTAGGTGGGGTAGCAGCGCCCCCCGCGGATGCTGGCGCGCACGTAGTCATACAGCTCGTGCGAGGGGGCGAGGAGCCCCGGGCCCAGGTTGGTGCGACTGGGCTTTTCGGCGCGGTAGACGATCTGGCGGAAAATGGCATGCGAGTTGGAGGAGATGGTGGGCCTTTGGAAGATGTTGAAGTGGGCGTGGGGCAGTCCGACCGAGTCGCGGATGAAGTGGGCGTAGGAGTCTTGCAGCTTGGCGACGAGCTCGGCGGTGACTAGGACGTCCAGAGCGCAGTAGTCGAGGGTCTCCTGGATGATGTCATACTTGAGCTGTCCCTTTTGTTTCCACAGCTCGCGGTTGAGAAGGAACTCTTCGCGGTCCTTCCAGTACTCTTCGAGGGGGAACCCGTCCTGATCTGCACGGTAAGAGCCTAGCATGTAGAACTGGTTGACGGCCTTGTAGGCGCAGCAGCCCTTCTCCACGGGGAGGGCGTAGGCCTGGGCGGCCTTGCGCAGGGAGGTGTGCGTGAGGGCGAAAGTGTCCCTGACCATGACCTTGAGGAACTGGTGCTTGAAGTCGATATCGTCGCAGCCCCCCTGCTCCCAGAGCTGGAAGTCCGTGCGCTTCTTGTAGGCGGGGTTGGGCAAAGCGAAAGTAACATCGTTGAAGAGGATCTTGCCCGCGCGGGGCATAAAGTTGCGAGTGATGCGGAAAGGTTGGGGCACCTCGGCCCGGTTGTTGATGACCTGGGCGGCGAGCACGATCTCGTCGAAGCCGTTGATGTTGTGGCCCACGATGTAGAGTTCCACGAATCGCGGACGGCCCTTGACGTGGGGCAGTTTCTTGAGCTCCTCGTAGGTGAGCTCGTCGGGGTCGCTGAGCCCGTGCTGCTCGAGCGCCCAGTCGGCGAGATGGGGGTTGGCGCGGAGGAAGGAAGTCCAGAGATCCACGGCCAGGGCGGTTTGCAGACGGTCCCGGTACTGACGGAACTGCTGCCCGACGGCCATTTTTTCGGGGGTGACGCAGTAGAAGGTGCGGGGGTCCCCGTGCCAGCGATCCCATTTGAGCTGGAGGGCGAGATCGAGGGCGAGCTCGACGAGCCGGTCGTCCCCGGAGAGTTTCATGACCAGCATGAAGGGGACGAGCTGCTTGCCGAAGGACCCCATCCAGGTGTAGGTTTCCACATCGTAGGTGAGGAAGAGCCTTTCGGTGCGAGGATGCGAGCCGATGGGGAAGAACTGGATCTCCTGCCACCAATTGGAGGAATGGCTGTTGATGTGATGGAAGTAGAAATGCCGACGGCGCGCCGAACACTCGTGCTTGTGTTTATACAAGCGGCCACAGTGCTCGCAACGCTGCACGGGATGCACGTGCTGCACGAGCTGTACCTGAGTTCCTTTGACGAGGAATTTCAGTGGGAAGTGGAGTCGTGGCGCCTGCATCTCGTGCTGTACTACGTCGTGGTGGTCGGCCTGGCCCTCTTCTGCCTCGATGGTGGTCATGCTGACGAGCCCGCGCGGGAGGCAGGTCCAGACCTCGGCGCGAGCGGGTCGGAGAGCGAGGACGAGGGCGCGCAGGCCGGAGCTGTCCAGGGTCCTGAGACGCTGCGGAGTCAGGTCAGTGGGCAGCGGCGGCGCGCGGTTGACTTGCAGGAGTTTTTCCAGGGCGCGCGGGAGGTCCAGATGGTACTTGATCTCCACCGCGCCATTGGTGGCGACGTCGATGGCTTGCAGGGTCCCGTGCCCCTGGGGTGTGACCACCGTCCCCCGTTTCTTCTTGGGCGGCTGGGGCGACGGGGGCGGTGCCTCTTCCATGGTTAGAAGCGGCGGCGAGGACGCGCGCCGGGCGGCAGGGGCGGCTCGGGGCCCGGAGGCAGGGGCGGCAGGGGCACGTCGGCGCCGCGCGCGGGTAGGTTCTGGTACTGCGCCCGGAGAAGACTGGCGTGAGCGACGACGCGACGGTTGACGTCCTGGATCTGACGCCTCTGGGTGAAGGCCACGGGACCCGTGAGTTTGAACCTGAAAGAGAGTTCGACAGAATCAATCTCGGTATCGTTGACGGCGGCCTGCCGCAGGATCTCTTGCACGTCGCCCGAGTTGTCCTGGTAGGCGATCTCGGTCATGAACTGCTCGATCTCCTCCTCTTGAAGGTCTCCGCGGCCGGCGCGCTCCACGGTGGCCGCGAGGTCGTTGGAGATGCGGCCCATGAGCTGCGAGAAGGCGTTCATGCCCGCCTCGTTCCAGACGCGGCTGTAGACCACGACGCCCTCGGGATCGCGGGCGCGCATGACCACCTGGGCGAGGTTGAGCTCCACGTGGCGCGTGAAGACCGCGTAGTTGCAGAGGCGCTGGTAGAGGTAGTTGAGCGTGGTGGCGATGTGCTCGGTGACGAAGAAATACATGATCCAGCGGCGGAGCGGCATCTCGCTGACGTCGCCCAGCGCCTCCAAACGTTCCATGGCCTCGTAAAAGTCCACGGCGAAGTTGAAAAACTGGGAGTTGCGCGCCGAGACGGTCAACTCCTCCTCCAGAAGACGGATGAGCTCGGCGATGGTGGCGCGCACCTCGCGCTCGAAGGCCCCCGGGAGTTCCTCCACTTCCTCTTCTTCCTCCTCCACTAACATCTCTTCTACTTCCTCCTCAGGCGGCAGTGGTGGCGGGGGAGGGGGCCTGCGTCGCCGGCGGCGCACGGGCAGACGGTCGATGAAGCGCTCGATGGTCTCGCCGCGCCGGCGTCGCATGGTCTCGGTGACGGCGCGCCCGTCCTCGCGGGGCCGCAGCGTGAAGACGCCGCCGCGCATCTCCAGGTGGCCGGGGGGGTCCCCGTTGGGCAGGGAGAGGGCGCTGACGATGCATCTTATCAATTGCCCCGTAGGGACTCCGCGCAAGGACCTGAGCGTCTCGAGATCCACGGGATCTGAAAACCGCTGAACGAAGGCTTCGAGCCAGTCGCAGTCGCAAGGTAGGCTGAGCACGGTTTCTTCTGGCGGGTCATGTTGGTTGGGAGCGGGGCGGGCGATGCTGCTGGTGATGAAGTTGAAATAGGCGGTTCTGAGACGGCGGATGGTGGCGAGGAGCACCAGGTCTTTGGGCCCGGCTTGCTGGATGCGCAGACGGTCGGCCATGCCCCAGGCGTGGTCCTGACACCTGGCCAGGTCCTTGTAGTAGTCCTGCATGAGCCGCTCCACGGGCACCTCCTCCTCGCCCGCGCGGCCGTGCATGCGCGTGAGCCCGAAGCCGCGCTGGGGCTGGACGAGCGCCAGGTCGGCGACGACGCGCTCGGCGAGGATGGCTTGCTGGATCTGGGTGAGGGTGGTCTGGAAGTCATCAAAGTCGACGAAGCGGTGGTAGGCTCCGGTGTTGATGGTGTAGGAGCAGTTGGCCATGACGGACCAGTTGACGGTCTGGTGGCCCGGACGCACGAGCTCGTGGTACTTGAGGCGCGAGTAGGCGCGCGTGTCGAAGATGTAGTCGTTGCAGGTGCGCACCAGGTACTGGTAGCCGATGAGGAAGTGCGGCGGCGGCTGGCGGTAGAGCGGCCATCGCTCGGTGGCGGGGGCGCCGGGCGCGAGGTCCTCGAGCATGGTGCGGTGGTAGCCGTAGATGTACCTGGACATCCAGGTGATGCCGGCGGCGGTGGTGGAGGCGCGCGGGAACTCGCGGACGCGGTTCCAGATGTTGCGCAGCGGCAGGAAGTAGTTCATGGTGGGCACGGTCTGGCCCGTGAGGCGCGCGCAGTCGTGGATGCTCTATACGGGCAAAAACGAAAGCGGTCAGCGGCTCGACTCCGTGGCCTGGAGGCTAAGCGAACGGGTTGGGCTGCGCGTGTACCCCGGTTCGAATCTCGAATCAGGCTGGAGCCGCAGCTAACGTGGTATTGGCACTCCCGTCTCGACCCAAGCCTGCACCAACCCTCCAGGATACGGAGGCGGGTCGTTTTGCAACTTTTTTTTGGAGGCCGGATGAGACTAGTAAGCGCGGAAAGCGGCCGACCGCGATGGCTCGCTGCCGTAGTCTGGAGAAGAATCGCCAGGGTTGCGTTGCGGTGTGCCCCGGTTCGAGGCCGGCCGGATTCCGCGGCTAACGAGGGCGTGGCTGCCCCGTCGTTTCCAAGACCCCATAGCCAGCCGACTTCTCCAGTTACGGAGCGAGCCCCTCTTTTGTTTTGTTTGTTTTTGCCAGATGCATCCCGTACTGCGGCAGATGCGCCCCCACCACCCTCCACCGCAACAACAGCCCCCTCCACAGCCGGCGCTTCTGCCCCCGCCCCAGCAGCAACTTCCAGCCACGACCGCCGCGGCCGCCGTGAGCGGGGCTGGACAGAGTTATGATCACCAGCTGGCCTTGGAAGAGGGCGAGGGGCTGGCGCGCCTGGGGGCGTCGTCGCCGGAGCGGCACCCGCGCGTGCAGATGAAAAGGGACGCTCGCGAGGCCTACGTGCCCAAGCAGAACCTGTTCAGAGACAGGAGCGGCGAGGAGCCCGAGGAGATGCGCGCGGCCCGGTTCCACGCGGGGCGGGAGCTGCGGCGCGGCCTGGACCGAAAGAGGGTGCTGAGGGACGAGGATTTCGAGGCGGACGAGCTGACGGGGATCAGCCCCGCGCGCGCGCACGTGGCCGCGGCCAACCTGGTCACGGCGTACGAGCAGACCGTGAAGGAGGAGAGCAACTTCCAAAAATCCTTCAACAACCACGTGCGCACCCTGATCGCGCGCGAGGAGGTGACCCTGGGCCTGATGCACCTGTGGGACCTGCTGGAGGCCATCGTGCAGAACCCCACCAGCAAGCCGCTGACGGCGCAGCTGTTCCTGGTGGTGCAGCATAGTCGGGACAACGAAGCGTTCAGGGAGGCGCTGCTGAATATCACCGAGCCCGAGGGCCGCTGGCTCCTGGACCTGGTGAACATTCTGCAGAGCATCGTGGTGCAGGAGCGCGGGCTGCCGCTGTCCGAGAAGCTGGCGGCCATCAACTTCTCGGTGCTGAGTTTGGGCAAGTACTACGCTAGGAAGATCTACAAGACCCCGTACGTGCCCATAGACAAGGAGGTGAAGATCGACGGGTTTTACATGCGCATGACCCTGAAAGTGCTGACCCTGAGCGACGATCTGGGGGTGTACCGCAACGACAGGATGCACCGTGCGGTGAGCGCCAGCAGGCGGCGCGAGCTGAGCGACCAGGAGCTGATGCATAGTCTGCAGCGGGCCCTGACCGGGGCCGGGACCGAGGGGGAGAGCTACTTTGACATGGGCGCGGACCTGCACTGGCAGCCCAGCCGCCGGGCCTTGGAGGCGGCGGCAGGACCCTACGTAGAAGAGGTGGACGATGAGGTGGACGAGGAGGGCGAGTACCTGGAAGACTGATGGCGCGACCGTATTTTTGCTAGATGCAACAACAACAGCCACCTCCTGATCCCGCGATGCGGGCGGCGCTGCAGAGCCAGCCGTCCGGCATTAACTCCTCGGACGATTGGACCCAGGCCATGCAACGCATCATGGCGCTGACGACCCGCAACCCCGAAGCCTTTAGACAGCAGCCCCAGGCCAACCGGCTCTCGGCCATCCTGGAGGCCGTGGTGCCCTCGCGCTCCAACCCCACGCACGAGAAGGTCCTGGCCATCGTGAACGCGCTGGTGGAGAACAAGGCCATCCGCGGCGACGAGGCCGGCCTGGTGTACAACGCGCTGCTGGAGCGCGTGGCCCGCTACAACAGCACCAACGTGCAGACCAACCTGGACCGCATGGTGACCGACGTGCGCGAGGCCGTGGCCCAGCGCGAGCGGTTCCACCGCGAGTCCAACCTGGGATCCATGGTGGCGCTGAACGCCTTCCTCAGCACCCAGCCCGCCAACGTGCCCCGGGGCCAGGAGGACTACACCAACTTCATCAGCGCCCTGCGCCTGATGGTGACCGAGGTGCCCCAGAGCGAGGTGTACCAGTCCGGGCCGGACTACTTCTTCCAGACCAGTCGCCAGGGCTTGCAGACCGTGAACCTGAGCCAGGCTTTCAAGAACTTGCAGGGCCTGTGGGGCGTGCAGGCCCCGGTCGGGGACCGCGCGACGGTGTCGAGCCTGCTGACGCCGAACTCGCGCCTGCTGCTGCTGCTGGTGGCCCCCTTCACGGACAGCGGCAGCATCAACCGCAACTCGTACCTGGGCTACCTGATTAACCTGTACCGCGAGGCCATCGGCCAGGCGCACGTGGACGAGCAGACCTACCAGGAGATCACCCACGTGAGCCGCGCCCTGGGCCAGGACGACCCGGGCAACCTGGAAGCCACCCTGAACTTTTTGCTGACCAACCGGTCGCAGAAGATCCCGCCCCAGTACGCGCTCAGCACCGAGGAGGAGCGCATCCTGCGTTACGTGCAGCAGAGCGTGGGCCTGTTCCTGATGCAGGAGGGGGCCACCCCCAGCGCCGCGCTCGACATGACCGCGCGCAACATGGAGCCCAGCATGTACGCCAGCAACCGCCCGTTCATCAATAAACTGATGGACTACTTGCATCGGGCGGCCGCCATGAACTCTGACTATTTCACCAACGCCATCCTGAATCCCCACTGGCTCCCGCCGCCGGGGTTCTACACGGGCGAGTACGACATGCCCGACCCCAATGACGGGTTCCTGTGGGACGATGTGGACAGCAGCGTGTTCTCCCCCCGACCGGGTGCTAACGAGCGCCCCTTGTGGAAGAAGGAAGGCAGCGACCGACGCCCGTCCTCGGCGCTGTCCGGCCGCGAGGGTGCTGCCGCGGCGGTGCCCGAGGCCGCCAGTCCTTTCCCGAGCTTGCCCTTCTCGCTGAACAGTATCCGCAGCAGCGAGCTGGGCAGGATCACGCGCCCGCGCTTGCTGGGCGAAGAGGAGTACTTGAATGACTCGCTGTTGAGACCCGAGCGGGAGAAGAACTTCCCCAATAACGGGATAGAAAGCCTGGTGGACAAGATGAGCCGCTGGAAGACGTATGCGCAGGAGCACAGGGACGATCCCCGGGCGTCGCAGGGGGCCACGAGCCGGGGCAGCGCCGCCCGTAAACGCCGGTGGCACGACAGGCAGCGGGGACAGATGTGGGACGATGAGGACTCCGCCGACGACAGCAGCGTGTTGGACTTGGGTGGGAGTGGTAACCCGTTCGCTCACCTGCGCCCCCGTATCGGGCGCATGATGTAAGAGAAACCGAAAATAAATGATACTCACCAAGGCCATGGCGACCAGCGTGCGTTCGTTTCTTCTCTGTTGTTGTTGTATCTAGTATGATGAGGCGTGCGTACCCGGAGGGTCCTCCTCCCTCGTACGAGAGCGTGATGCAGCAGGCGATGGCGGCGGCGGCGATGCAGCCCCCGCTGGAGGCTCCTTACGTGCCCCCGCGGTACCTGGCGCCTACGGAGGGGCGGAACAGCATTCGTTACTCGGAGCTGGCACCCTTGTACGATACCACCCGGTTGTACCTGGTGGACAACAAGTCGGCGGACATCGCCTCGCTGAACTACCAGAACGACCACAGCAACTTCCTGACCACCGTGGTGCAGAACAATGACTTCACCCCCACGGAGGCCAGCACCCAGACCATCAACTTTGACGAGCGCTCGCGGTGGGGCGGCCAGCTGAAAACCATCATGCACACCAACATGCCCAACGTGAACGAGTTCATGTACAGCAACAAGTTCAAGGCGCGGGTGATGGTCTCCCGCAAGACCCCCAATGGGGTGACAGTGACAGAGGATTATGATGGTAGTCAGGATGAGCTGAAGTATGAATGGGTGGAATTTGAGCTGCCCGAAGGCAACTTCTCGGTGACCATGACCATCGACCTGATGAACAACGCCATCATCGACAATTACTTGGCGGTGGGGCGGCAGAACGGGGTGCTGGAGAGCGACATCGGCGTGAAGTTCGACACTAGGAACTTCAGGCTGGGCTGGGACCCCGTGACCGAGCTGGTCATGCCCGGGGTGTACACCAACGAGGCTTTCCATCCCGATATTGTCTTGCTGCCCGGCTGCGGGGTGGACTTCACCGAGAGCCGCCTCAGCAACCTGCTGGGCATTCGCAAGAGGCAGCCCTTCCAGGAAGGCTTCCAGATCATGTACGAGGATCTGGAGGGGGGCAACATCCCCGCGCTCCTGGATGTCGACGCCTATGAGAAAAGCAAGGAGGATGCAGCAGCTGAAGCAACTGCAGCCGTAGCTACCGCCTCTACCGAGGTCAGGGGCGATAATTTTGCAAGCGCCGCAGCAGTGGCAGCGGCCGAGGCGGCTGAAACCGAAAGTAAGATAGTCATTCAGCCGGTGGAGAAGGATAGCAAGAACAGGAGCTACAACGTACTACCGGACAAGATAAACACCGCCTACCGCAGCTGGTACCTAGCCTACAACTATGGCGACCCCGAGAAGGGCGTGCGCTCCTGGACGCTGCTCACCACCTCGGACGTCACCTGCGGCGTGGAGCAAGTCTACTGGTCGCTGCCCGACATGATGCAAGACCCGGTCACCTTCCGCTCCACGCGTCAAGTTAGCAACTACCCGGTGGTGGGCGCCGAGCTCCTGCCCGTCTACTCCAAGAGCTTCTTCAACGAGCAGGCCGTCTACTCGCAGCAGCTGCGCGCCTTCACCTCGCTTACGCACGTCTTCAACCGCTTCCCCGAGAACCAGATCCTCGTCCGCCCGCCCGCGCCCACCATTACCACCGTCAGTGAAAACGTTCCTGCTCTCACAGATCACGGGACCCTGCCGCTGCGCAGCAGTATCCGGGGAGTCCAGCGCGTGACCGTTACTGACGCCAGACGCCGCACCTGCCCCTACGTCTACAAGGCCCTGGGCATAGTCGCGCCGCGCGTCCTCTCGAGCCGCACCTTCTAAATGTCCATTCTCATCTCGCCCAGTAATAACACCGGTTGGGGCCTGCGCGCGCCCAGCAAGATGTACGGAGGCGCTCGCCAACGCTCCACGCAACACCCCGTGCGCGTGCGCGGGCACTTCCGCGCTCCCTGGGGCGCCCTCAAGGGCCGCGTGCGGTCGCGCACCACCGTCGACGACGTGATCGACCAGGTGGTGGCCGACGCGCGCAACTACACCCCCGCCGCCGCGCCCGTCTCCACCGTGGACGCCGTCATCGACAGCGTGGTGGCCGACGCGCGCCGGTACGCCCGCGCCAAGAGCCGGCGGCGGCGCATCGCCCGGCGGCACCGGAGCACCCCCGCCATGCGCGCGGCGCGAGCCTTGCTGCGCAGGGCCAGGCGCACGGGACGCAGGGCCATGCTCAGGGCGGCCAGACGCGCGGCTTCAGGCGCCAGCGCCGGCAGGACCCGGAGACGCGCGGCCACGGCGGCGGCAGCGGCCATCGCCAGCATGTCCCGCCCGCGGCGAGGGAACGTGTACTGGGTGCGCGACGCCGCCACCGGTGTGCGCGTGCCCGTGCGCACCCGCCCCCCTCGCACTTGAAGATGTTCACTTCGCGATGTTGATGTGTCCCAGCGGCGAGGAGGATGTCCAAGCGCAAATTCAAGGAAGAGATGCTCCAGGTCATCGCGCCTGAGATCTACGGCCCTGCGGTGGTGAAGGAGGAAAGAAAGCCCCGCAAAATCAAGCGGGTCAAAAAGGACAAAAAGGAAGAAGAAAGTGATGTGGACGGATTGGTGGAGTTTGTGCGCGAGTTCGCCCCCCGGCGGCGCGTGCAGTGGCGCGGGCGGAAGGTGCAACCGGTGCTGAGACCCGGCACCACCGTGGTCTTCACGCCCGGCGAGCGCTCCGGCACCGCTTCCAAGCGCTCCTACGACGAGGTGTACGGGGATGATGATATTCTGGAGCAGGCGGCCGAGCGCCTGGGCGAGTTTGCTTACGGCAAGCGCAGCCGTTCCGCACCGAAGGAAGAGGCGGTGTCCATCCCGCTGGACCACGGCAACCCCACGCCGAGCCTCAAGCCCGTGACCTTGCAGCAGGTGCTGCCGACCGCGGCGCCGCGCCGGGGGTTCAAGCGCGAGGGCGAGGATCTGTACCCCACCATGCAGCTGATGGTGCCCAAGCGCCAGAAGCTGGAAGACGTGCTGGAGACCATGAAGGTGGACCCGGACGTGCAGCCCGAGGTCAAGGTGCGGCCCATCAAGCAGGTGGCCCCGGGCCTGGGCGTGCAGACCGTGGACATCAAGATTCCCACGGAGCCCATGGAAACGCAGACCGAGCCCATGATCAAGCCCAGCACCAGCACCATGGAGGTGCAGACGGATCCCTGGATGCCATCGGCTCCTAGTCGAAGACCCCGGCGCAAGTACGGCGCGGCCAGCCTGCTGATGCCCAACTACGCGCTGCATCCTTCCATCATCCCCACGCCGGGCTACCGCGGCACGCGCTTCTACCGCGGTCATACCAGCAGCCGCCGCCGCAAGACCACCACTCGCCGCCGCCGTCGCCGCACCGCCGCTGCAACCACCCCTGCCGCCCTGGTGCGGAGAGTGTACCGCCGCGGCCGCGCACCTCTGACCCTGCCGCGCGCGCGCTACCACCCGAGCATCGCCATTTAAACTTTCGCCTGCTTTGCAGATCAATGGCCCTCACATGCCGCCTTCGCGTTCCCATTACGGGCTACCGAGGAAGAAAACCGCGCCGTAGAAGGCTGGCGGGGAACGGGATGCGTCGCCACCACCACCGGCGGCGGCGCGCCATCAGCAAGCGGTTGGGGGGAGGCTTCCTGCCCGCGCTGATCCCCATCATCGCCGCGGCGATCGGGGCGATCCCCGGCATTGCTTCCGTGGCGGTGCAGGCCTCTCAGCGCCACTGAGACACACTTGGAAACATCTTGTAATAAACCAATGGACTCTGACGCTCCTGGTCCTGTGATGTGTTTTCGTAGACAGATGGAAGACATCAATTTTTCGTCCCTGGCTCCGCGACACGGCACGCGGCCGTTCATGGGCACCTGGAGCGACATCGGCACCAGCCAACTGAACGGGGGCGCCTTCAATTGGAGCAGTCTCTGGAGCGGGCTTAAGAATTTCGGGTCCACGCTTAAAACCTATGGCAGCAAGGCGTGGAACAGCACCACAGGGCAGGCGCTGAGGGATAAGCTGAAAGAGCAGAACTTCCAGCAGAAGGTGGTCGATGGGCTCGCCTCGGGCATCAACGGGGTGGTGGACCTGGCCAACCAGGCCGTGCAGCGGCAGATCAACAGCCGCCTGGACCCGGTGCCGCCCGCCGGCTCCGTGGAGATGCCGCAGGTGGAGGAGGAGCTGCCTCCCCTGGACAAGCGGGGCGAGAAGCGACCCCGCCCCGATGCGGAGGAGACGCTGCTGACGCACACGGACGAGCCGCCCCCGTACGAGGAGGCGGTGAAACTGGGTCTGCCCACCACGCGGCCCATCGCGCCCCTGGCCACCGGGGTGCTGAAACCCGAAAAGCCCGCGACCCTGGACTTGCCTCCTCCCCAGCCTTCCCGCCCCTCTACAGTGGCTAAGCCCCTGCCGCCGGTGGCCGTGGCCCGCGCGCGACCCGGGGGCACCGCCCGCCCTCATGCGAACTGGCAGAGCACTCTGAACAGCATCGTGGGTCTGGGAGTGCAGAGTGTGAAGCGCCGCCGCTGCTATTAAACCTACCGTAGCGCTTAACTTGCTTGTCTGTGTGTGTATGTATTATGTCGCCGCCGCCGCTGTCCACCAGAAGGAGGAGTGAAGAGGCGCGTCGCCGAGTTGCAAGATGGCCACCCCATCGATGCTGCCCCAGTGGGCGTACATGCACATCGCCGGACAGGACGCTTCGGAGTACCTGAGTCCGGGTCTGGTGCAGTTTGCCCGCGCCACAGACACCTACTTCAGTCTGGGGAACAAGTTTAGGAACCCCACGGTGGCGCCCACGCACGATGTGACCACCGACCGCAGCCAGCGGCTGACGCTGCGCTTCGTGCCCGTGGACCGCGAGGACAACACCTACTCGTACAAAGTGCGCTACACGCTGGCCGTGGGCGACAACCGCGTGCTGGACATGGCCAGCACCTACTTTGACATCCGCGGCGTGCTGGATCGGGGCCCTAGCTTCAAACCCTACTCCGGCACCGCCTACAACAGTCTGGCCCCCAAGGGAGCACCCAACACTTGTCAGTGGACATATAAAGCCGATGGTGAAACTGCCACAGAAAAAACCTATACATATGGAAATGCACCCGTGCAGGGCATTAACATCACAAAAGATGGTATTCAACTTGGAACTGACACCGATGATCAGCCAATCTACGCAGATAAAACCTATCAGCCTGAACCTCAAGTGGGTGATGCTGAATGGCATGACATCACTGGTACTGATGAAAAGTATGGAGGCAGAGCTCTTAAGCCTGATACCAAAATGAAGCCTTGTTATGGTTCTTTTGCCAAGCCTACTAATAAAGAAGGAGGTCAGGCAAATGTGAAAACAGGAACAGGCACTACTAAAGAATATGACATAGACATGGCTTTCTTTGACAACAGAAGTGCGGCTGCTGCTGGCCTAGCTCCAGAAATTGTTTTGTATACTGAAAATGTGGATTTGGAAACTCCAGATACCCATATTGTATACAAAGCAGGCACAGATGACAGCAGCTCTTCTATTAATTTGGGTCAGCAAGCCATGCCCAACAGACCTAACTACATTGGTTTCAGAGACAACTTTATCGGGCTCATGTACTACAACAGCACTGGCAATATGGGGGTGCTGGCCGGTCAGGCTTCTCAGCTGAATGCTGTGGTTGACTTGCAAGACAGAAACACCGAGCTGTCCTACCAGCTCTTGCTTGACTCTCTGGGTGACAGAACCCGGTATTTCAGTATGTGGAATCAGGCGGTGGACAGCTATGATCCTGATGTGCGCATTATTGAAAATCATGGTGTGGAGGATGAACTTCCCAACTATTGTTTCCCTCTGGATGCTGTTGGCAGAACAGATACTTATCAGGGAATTAAGGCTAATGGAACTGATCAAACCACATGGACCAAAGATGACAGTGTCAATGATGCTAATGAGATAGGCAAGGGTAATCCATTCGCCATGGAAATCAACATCCAAGCCAACCTGTGGAGGAACTTCCTCTACGCCAACGTGGCCCTGTACCTGCCCGACTCTTACAAGTACACGCCGGCCAATGTTACCCTGCCCACCAACACCAACACCTACGATTACATGAACGGCCGGGTGGTGGCGCCCTCGCTGGTGGACTCCTACATCAACATCGGGGCGCGCTGGTCGCTGGATCCCATGGACAACGTGAACCCCTTCAACCACCACCGCAATGCGGGGCTGCGCTACCGCTCCATGCTCCTGGGCAACGGGCGCTACGTGCCCTTCCACATCCAGGTGCCCCAGAAATTTTTCGCCATCAAGAGCCTCCTGCTCCTGCCCGGGTCCTACACCTACGAGTGGAACTTCCGCAAGGACGTCAACATGATCCTGCAGAGCTCCCTCGGCAACGACCTGCGCACGGACGGGGCCTCCATCTCCTTCACCAGCATCAACCTCTACGCCACCTTCTTCCCCATGGCGCACAACACGGCCTCCACGCTCGAGGCCATGCTGCGCAACGACACCAACGACCAGTCCTTCAACGACTACCTCTCGGCGGCCAACATGCTCTACCCCATCCCGGCCAACGCCACCAACGTGCCCATCTCCATCCCCTCGCGCAACTGGGCCGCCTTCCGCGGCTGGTCCTTCACGCGTCTCAAGACCAAGGAGACGCCCTCGCTGGGCTCCGGGTTCGACCCCTACTTCGTCTACTCGGGCTCCATCCCCTACCTCGACGGCACCTTCTACCTCAACCACACCTTCAAGAAGGTCTCCATCACCTTCGACTCCTCCGTCAGCTGGCCCGGCAACGACCGGCTCCTGACGCCCAACGAGTTCGAAATCAAGCGCACCGTCGACGGCGAGGGCTACAACGTGGCCCAGTGCAACATGACCAAGGACTGGTTCCTGGTCCAGATGCTGGCCCACTACAACATCGGCTACCAGGGCTTCTACGTGCCCGAGGGCTACAAGGACCGCATGTACTCCTTCTTCCGCAACTTCCAGCCCATGAGCCGCCAGGTGGTGGACGAGGTCAACTACAAGGACTACCAGGCCGTCACCCTGGCCTACCAGCACAACAACTCGGGCTTCGTCGGCTACCTCGCGCCCACCATGCGCCAGGGCCAGCCCTACCCCGCCAACTACCCCTACCCGCTCATCGGCAAGAGCGCCGTCACCAGCGTCACCCAGAAAAAGTTCCTCTGCGACAGGGTCATGTGGCGCATCCCCTTCTCCAGCAACTTCATGTCCATGGGCGCGCTCACCGACCTCGGCCAGAACATGCTCTATGCCAACTCCGCCCACGCGCTAGACATGAATTTCGAAGTCGACCCCATGGATGAGTCCACCCTTCTCTATGTTGTCTTCGAAGTCTTCGACGTCGTCCGAGTGCACCAGCCCCACCGCGGCGTCATCGAGGCCGTCTACCTGCGCACCCCCTTCTCGGCCGGTAACGCCACCACCTAAGCTCTTGCTTCTTGCAAGCCATGGCCGCGGGCTCCGGCGAGCAGGAGCTCAGGGCCATCATCCGCGACCTGGGCTGCGGGCCCTACTTCCTGGGCACCTTCGATAAGCGCTTCCCGGGATTCATGGCCCCGCACAAGCTGGCCTGCGCCATCGTCAACACGGCCGGCCGCGAGACCGGGGGCGAGCACTGGCTGGCCTTCGCCTGGAACCCGCGCTCGAACACCTGCTACCTCTTCGACCCCTTCGGGTTCTCGGACGAGCGCCTCAAGCAGATCTACCAGTTCGAGTACGAGGGCCTGCTGCGCCGCAGCGCCCTGGCCACCGAGGACCGCTGCGTCACCCTGGAAAAGTCCACCCAGACCGTGCAGGGTCCGCGCTCGGCCGCCTGCGGGCTCTTCTGCTGCATGTTCCTGCACGCCTTCGTGCACTGGCCCGACCGCCCCATGGACAAGAACCCCACCATGAACTTGCTGACGGGGGTGCCCAACGGCATGCTCCAGTCGCCCCAGGTGGAACCCACCCTGCGCCGCAACCAGGAGGCGCTCTACCGCTTCCTCAACTCCCACTCCGCCTACTTTCGCTCCCACCGCGCGCGCATCGAGAAGGCCACCGCCTTCGACCGCATGAATCAAGACATGTAAACCGTGTGTGTATGTTAAATGTCTTTAATAAACAGCACTTTCATGTTACACATGCATCTGAGATGATTTATTTAGAAATCGAAAGGGTTCTGCCGGGTCTCGGCATGGCCCGCGGGCAGGGACACGTTGCGGAACTGGTACTTGGCCAGCCACTTGAACTCGGGGATCAGCAGTTTGGGCAGCGGGGTGTCGGGGAAGGAGTCGGTCCACAGCTTCCGCGTCAGTTGCAGGGCGCCCAGCAGGTCGGGCGCGGAGATCTTGAAATCGCAGTTGGGACCCGCGTTCTGCGCGCGGGAGTTGCGGTACACGGGGTTGCAGCACTGGAACACCATCAGGGCCGGGTGCTTCACGCTCGCCAGCACCGTCGCGTCGGTGATGCTCTCCACGTCGAGGTCCTCGGCGTTGGCCATCCCGAAGGGGGTCATCTTGCAGGTCTGCCTTCCCATGGTGGGCACGCACCCGGGCTTGTGGTTGCAATCGCAGTGCAGGGGGATCAGCATCATCTGGGCCTGGTCGGCGTTCATCCCCGGGTACATGGCCTTCATGAAAGCCTCCAATTGCCTGAACGCCTGCTGGGCCTTGGCTCCCTCGGTGAAGAAGACCCCGCAGGACTTGCTAGAGAACTGGTTGGTGGCGCACCCGGCGTCGTGCACGCAGCAGCGCGCGTCGTTGTTGGCCAGCTGCACCACGCTGCGCCCCCAGCGGTTCTGGGTGATCTTGGCCCGGTCGGGGTTCTCCTTCAGCGCGCGCTGCCCGTTCTCGCTCGCCACATCCATCTCGATCATGTGCTCCTTCTGGATCATGGTGGTCCCGTGCAGGCACCGCAGCTTGCCCTCGGCCTCGGTGCACCCGTGCAGCCACAGCGCGCACCCGGTGCACTCCCAGTTCTTGTGGGCGATCTGGGAATGCGCGTGCACGAAGCCCTGCAGGAAGCGGCCCATCATGGTGGTCAGGGTCTTGTTGCTAGTGAAGGTCAGCGGAATGCCGCGGTGCTCCTCGTTGATGTACAGGTGGCAGATGCGGCGGTACACCTCGCCCTGCTCGGGCATCAGCTGGAAGTTGGCTTTCAGGTCGGTCTCCACGCGGTAGCGGTCCATCAGCATAGTCATGATTTCCATACCCTTCTCCCAGGCCGAGACGATGGGCAGGCTCATAGGGTTCTTCACCATCATCTTAGCGCTAGCAGCCGCGGCCAGGGGGTCGCTCTCGTCCAGGGTCTCAAAGCTCCGCTTGCCGTCCTTCTCGGTGATCCGCACCGGGGGGTAGCTGAAGCCCACGGCCGCCAGCTCCTCCTCGGCCTGTCTTTCGTCCTCGCTGTCCTGGCTGACGTCCTGCAGGACCACATGCTTGGTCTTGCGGGGTTTCTTCTTGGGCGGCAGCGGCGGCGGAGATGTTGGAGATGGCGAGGGGGAGCGCGAGTTCTCGCTCACCACTACTATCTCTTCCTCTTCTTGGTCCGAGGCCACGCGGCGGTAGGTATGTCTCTTCGGGGGCAGAGGCGGAGGCGACGGGCTCTCGCCGCCGCGACTTGGCGGATGGCTGGCAGAGCCCCTTCCGCGTTCGGGGGTGCGCTCCCGGCGGCGCTCTGACTGACTTCCTCCGCGGCCGGCCATTGTGTTCTCCTAGGGAGGAACAACAAGCATGGAGACTCAGCCATCGCCAACCTCGCCATCTGCCCCCACCGCCGACGAGAAGCAGCAGCAGCAGAATGAAAGCTTAACCGCCCCGCCGCCCAGCCCCGCCACCTCCGACGCGGCCGTCCCAGACATGCAAGAGATGGAGGAATCCATCGAGATTGACCTGGGCTATGTGACGCCCGCGGAGCACGAGGAGGAGCTGGCAGTGCGCTTTTCACAAGAAGAGATACACCAAGAACAGCCAGAGCAGGAAGCAGAGAATGAGCAGAGTCAGGCTGGGCTCGAGCATGACGGCGACTACCTCCACCTGAGCGGGGGGGAGGACGCGCTCATCAAGCATCTGGCCCGGCAGGCCACCATCGTCAAGGATGCGCTGCTCGACCGCACCGAGGTGCCCCTCAGCGTGGAGGAGCTCAGCCGCGCCTACGAGTTGAACCTCTTCTCGCCGCGCGTGCCCCCCAAGCGCCAGCCCAATGGCACCTGCGAGCCCAACCCGCGCCTCAACTTCTACCCGGTCTTCGCGGTGCCCGAGGCCCTGGCCACCTACCACATCTTTTTCAAGAACCAAAAGATCCCCGTCTCCTGCCGCGCCAACCGCACCCGCGCCGACGCCCTTTTCAACCTGGGTCCCGGCGCCCGCCTACCTGATATCGCCTCCTTGGAAGAGGTTCCCAAGATCTTCGAGGGTCTGGGCAGCGACGAGACTCGGGCCGCGAACGCTCTGCAAGGAGAAGGAGGAGAGCATGAGCACCACAGCGCCCTGGTCGAGTTGGAAGGCGACAACGCGCGGCTGGCGGTGCTCAAACGCACGGTCGAGCTGACCCATTTCGCCTACCCGGCTCTGAACCTGCCCCCCAAAGTCATGAGCGCGGTCATGGACCAGGTGCTCATCAAGCGCGCGTCGCCCATCTCCGAGGACGAGGGCATGCAAGACTCCGAGGAGGGCAAGCCCGTGGTCAGCGACGAGCAGCTGGCCCGGTGGCTGGGTCCTAATGCTAGTCCCCAGAGTTTGGAAGAGCGGCGCAAACTCATGATGGCCGTGGTCCTGGTGACCGTGGAGCTGGAGTGCCTGCGCCGCTTCTTCGCCGACGCGGAGACCCTGCGCAAGGTCGAGGAGAACCTGCACTACCTCTTCAGGCACGGGTTCGTGCGCCAGGCCTGCAAGATCTCCAACGTGGAGCTGACCAACCTGGTCTCCTACATGGGCATCTTGCACGAGAACCGCCTGGGGCAGAACGTGCTGCACACCACCCTGCGCGGGGAGGCCCGGCGCGACTACATCCGCGACTGCGTCTACCTCTACCTCTGCCACACCTGGCAGACGGGCATGGGCGTGTGGCAGCAGTGTCTGGAGGAGCAGAACCTGAAAGAGCTCTGCAAGCTCCTGCAGAAGAACCTCAAGGGTCTGTGGACCGGGTTCGACGAGCGCACCACCGCCTCGGACCTGGCCGACCTCATTTTCCCCGAGCGCCTCAGGCTGACGCTGCGCAACGGCCTGCCCGACTTTATGAGCCAAAGCATGTTGCAAAACTTTCGCTCTTTCATCCTCGAACGCTCCGGAATCCTGCCCGCCACCTGCTCCGCGCTGCCCTCGGACTTCGTGCCGCTGACCTTCCGCGAGTGCCCCCCGCCGCTGTGGAGCCACTGCTACCTGCTGCGCCTGGCCAACTACCTGGCCTACCACTCGGACGTGATCGAGGACGTCAGCGGCGAGGGCCTGCTCGAGTGCCACTGCCGCTGCAACCTCTGCACGCCGCACCGCTCCCTGGCCTGCAACCCCCAGCTGCTGAGCGAGACCCAGATCATCGGCACCTTCGAGTTGCAAGGGCCCAGCGAAGGCGAGGGTTCAGCCGCCAAGGGGGGTCTGAAACTCACCCCGGGGCTGTGGACCTCGGCCTACTTGCGCAAGTTCGTGCCCGAGGACTACCATCCCTTCGAGATCAGGTTCTACGAGGACCAATCCCATCCGCCCAAGGCCGAGCTGTCGGCCTGCGTCATCACCCAGGGGGCGATCCTGGCCCAATTGCAAGCCATCCAGAAATCCCGCCAAGAATTCTTGCTGAAAAAGGGCCGCGGGGTCTACCTCGACCCCCAGACCGGTGAGGAGCTCAACCCCGGCTTCCCCCAGGATGCCCCGAGGAAACAAGAAGCTGAAAGTGGAGCTGCCGCCCGTGGAGGATTTGGAGGAAGACTGGGAGAACAGCAGTCAGGCAGAGGAGGAGGAGATGGAGGAAGACTGGGACAGCACTCAGGCAGAGGAGGACAGCCTGCAAGACAGTCTGGAGGAAGACGAGGAGGAGGCAGAGGAGGAGGTGGAAGAAGCAGCCGCCGCCAGACCGTCGTCCTCGGCGGGGGAGAAAGCAAGCAGCACGGATACCATCTCCGCTCCGGGTCGGGGTCCCGCTCGACCACACAGTAGATGGGACGAGACCGGACGATTCCCGAACCCCACCACCCAGACCGGTAAGAAGGAGCGGCAGGGATACAAGTCCTGGCGGGGGCACAAAAACGCCATCGTCTCCTGCTTGCAGGCCTGCGGGGGCAACATCTCCTTCACCCGGCGCTACCTGCTCTTCCACCGCGGGGTGAACTTTCCCCGCAACATCTTGCATTACTACCGTCACCTCCACAGCCCCTACTACTTCCAAGAAGAGGCAGCAGCAGCAGAAAAAGACCAGCAGAAAACCAGCAGCTAGAAAATCCACAGCGGCGGCAGCAGGTGGACTGAGGATCGCGGCGAACGAGCCGGCGCAAACCCGGGAGCTGAGGAACCGGATCTTTCCCACCCTCTATGCCATCTTCCAGCAGAGTCGGGGGCAGGAGCAGGAACTGAAAGTCAAGAACCGTTCTCTGCGCTCGCTCACCCGCAGTTGTCTGTATCACAAGAGCGAAGACCAACTTCAGCGCACTCTCGAGGACGCCGAGGCTCTCTTCAACAAGTACTGCGCGCTCACTCTTAAAGAGTAGCCCGCGCCCGCCCAGTCGCAGAAAAAGGCGGGAATTACGTCACCTGTGCCCTTCGCCCTAGCCGCCTCCACCCATCATCATGAGCAAAGAGATTCCCACGCCTTACATGTGGAGCTACCAGCCCCAGATGGGCCTGGCCGCCGGTGCCGCCCAGGACTACTCCACCCGCATGAATTGGCTCAGCGCCGGGCCCGCGATGATCTCACGGGTGAATGACATCCGCGCCCACCGAAACCAGATACTCCTAGAACAGTCAGCGCTCACCGCCACGCCCCGCAATCACCTCAATCCGCGTAATTGGCCCGCCGCCCTGGTGTACCAGGAAATTCCCCAGCCCACGACCGTACTACTTCCGCGAGACGCCCAGGCCGAAGTCCAGCTGACTAACTCAGGTGTCCAGCTGGCGGGCGGCGCCACCCTGTGTCGTCACCGCCCCGCTCAGGGTATAAAGCGGCTGGTGATCCGGGGCAGAGGCACACAGCTCAACGACGAGGTGGTGAGCTCTTCGCTGGGTCTGCGACCTGACGGAGTCTTCCAACTCGCCGGATCGGGGAGATCTTCCTTCACGCCTCGTCAGGCCGTCCTGACTTTGGAGAGTTCGTCCTCGCAGCCCCGCTCGGGTGGCATCGGCACTCTCCAGTTCGTGGAGGAGTTCACTCCCTCGGTCTACTTCAACCCCTTCTCCGGCTCCCCCGGCCACTACCCGGACGAGTTCATCCCGAACTTCGACGCCATCAGCGAGTCGGTGGACGGCTACGATTGAATGTCCCATGGTGGCGCAGCTGACCTAGCTCGGCTTCGACACCTGGACCACTGCCGCCGCTTCCGCTGCTTCGCTCGGGATCTCGCCGAGTTTGCCTACTTTGAGCTGCCCGAGGAGCACCCTCAGGGCCCGGCCCACGGAGTGCGGATCGTCGTCGAAGGGGGCCTCGACTCCCACCTGCTTCGGATCTTCAGCCAGCGTCCGATCCTGGTCGAGCGCGAGCAAGGACAGACCCTTCTGACTCTGTACTGCATCTGCAACCACCCCGGCCTGCATGAAAGTCTTTGTTGTCTGCTGTGTACTGAGTATAATAAAAGCTGAGATCAGCGACTACTCCGGACTTCCGTGTGTTCCTGAATCCATCAACCAGTCTTTGTTCTTCACCGGGAACGAGACCGAGCTCCAGCTCCAGTGTAAGCCCCACAAGAAGTACCTCACCTGGCTGTTCCAGGGCTCCCCGATCGCCGTTGTCAACCACTGCGACAACGACGGAGTCCTGCTGAGCGGCCCTGCCAACCTTACTTTTTCCACCCGCAGAAGCAAGCTCCAGCTCTTCCAACCCTTCCTCCCCGGGACCTATCAGTGCGTCTCGGGACCCTGCCATCACACCTTCCACCTGATCCCGAATACCACAGCGTCGCTCCCCGCTACTAACAACCAAACTAACCTCCACCAACGCCACCGTCGCGACCTAGGCCTTTTAATTTTTTCTATCATTACCTCTGCTCTATGCAATTCTGACAATGAGGACGTTACTGTCGTTGTCGGATCAAATTATACACTGAAAGGTCCAGCGAAGGGTATGCTTTCGTGGTATTGCTATTTTGGATCTGACACTACAGAAACTGAATTATGCAATCTTAAGAATGGCAAAATTCAAAATTCTAAAATTAACAATTATATATGCAATGGTACTGATCTGATACTCCTCAATATCACGAAATCATATGCTGGCAGTTACACCTGCCCTGGAGATGATGCTGACAGTATGATTTTTTACAAAGTAACTGTTGTTGATCCCACTACTCCACCTCCACCCACCACAACTACTCACACCACACACACAGATCAAACCGCAGCAGAGGAGGCAGCAAAGTTAGCCTTGCAGGTCCAAGACAGTTCATTTGTTGGCATTACCCCTACACCTGATCAGCGGTGTCCGGGGCTGCTAGTCAGCGGCATTGTCGGTGTGCTTTCGGGATTAGCAGTCATAATCATCTGCATGTTCATTTTTGCTTGCTGCTATAGAAGGCTTTACCGACAAAAATCAGACCCACTGCTGAACCTCTATGTTTAATTTTTTCCAGAGTCATGAAGGCAGTTAGCGCTCTAGTTTTTTGTTCTTTGATTGGCATTGTTTTTTGCAATCCTATTCCTAAAGTTAGCTTTATTAAAGATGTGAATGTTACTGAGGGGGGCAATGTGACACTGGTAGGTGTAGAGGGTGCTGAAAACACCACCTGGACAAAATACCACCTCAATGGGTGGAAAGATATTTGCAATTGGAGTGTATTAGTTTATACATGTGAGGGAGTTAATCTTACCATTGTCAATGCCACCTCAGCTCAAAATGGTAGAATTCAAGGACAAAGTGTCAGTGTATCTAATGGGTATTTTACCCAACATACTTTTATCTATGACGTTAAAGTCATACCACTGCCTACGCCTAGCCCACCTAGCACTACCACACAGACAACCCACACTACACAGACAACCACATACAGTACATTAAATCAGCCTACCACCACTACAGCAGCAGAGGTTGCCAGCTCGTCTGGGGTCCGAGTGGCATTTTTGATGTTGGCCCCATCTAGCAGTCCCACTGCTAGTACCAATGAGCAGACTACTGAATTTTTGTCCACTGTCGAGAGCCACACCACAGCTACCTCCAGTGCCTTCTCTAGCACCGCCAATCTCTCCTCGCTTTCCTCTACACCAATCAGTCCCGCTACTACTCCTAGCCCCGCTCCTCTTCCCACTCCCCTGAAGCAAACAGACGGCGGCATGCAATGGCAGATCACCCTGCTCATTGTGATCGGGTTGGTCATCCTGGCCGTGTTGCTCTACTACATCTTCTGCCGCCGCATTCCCAACGCGCACCGCAAGCCGGTCTACAAGCCCATCATTGTCGGGCAGCCGGAGCCGCTTCAGGTGGAAGGGGGTCTAAGGAATCTTCTCTTCTCTTTTACAGTATGGTGATTGAACTATGATTCCTAGACAATTCTTGATCACTATTCTTATCTGCCTCCTCCAAGTCTGTGCCACCCTCGCTCTGGTGGCCAACGCCAGTCCAGACTGTATTGGGCCCTTCGCCTCCTACGTGCTCTTTGCCTTCACCACCTGCATCTGCTGCTGTAGCATAGTCTGCCTGCTTATCACCTTCTTCCAGTTCATTGACTGGATCTTTGTGCGCATCGCCTACCTGCGCCACCACCCCCAGTACCGCGACCAGCGAGTGGCGCGGCTGCTCAGGCTCCTCTGATAAGCATGCGGGCTCTGCTACTTCTCGCGCTTCTGCTGTTAGTGCTCCCCCGTCCCGTCGACCCCCGGTCCCCCACCCAGTCCCCCGAGGAGGTCCGCAAATGCAAATTCCAAGAACCCTGGAAATTCCTCAAATGCTACCGCCAAAAATCAGACATGCATCCCAGCTGGATCATGATCATTGGGATCGTGAACATTCTGGCCTGCACCCTCATCTCCTTTGTGATTTACCCCTGCTTTGACTTTGGTTGGAACTCGCCAGAGGCGCTCTATCTCCCGCCTGAACCTGACACACCACCACAGCAACCTCAGGCACACGCACTACCACCACTACAGCCTAGGCCACAATACATGCCCATATTAGACTATGAGGCCGAGCCACAGCGACCCATGCTCCCCGCTATTAGTTACTTCAATCTAACCGGCGGAGATGACTGACCCACTGGCCAACAACAACGTCAACGACCTTCTCCTGGACATGGACGGCCGCGCCTCGGAGCAGCGACTCGCCCAACTTCGCATTCGCCAGCAGCAGGAGAGAGCCGTCAAGGAGCTGCAGGATGCGGTGGCCATCCACCAGTGCAAGAGAGGCATCTTCTGCCTGGTGAAACAGGCCAAGATCTCCTACGAGGTCACTCCAAACGACCATCGCCTCTCCTACGAGCTCCTGCAGCAGCGCCAGAAGTTCACCTGCCTGGTCGGAGTCAACCCCATCGTCATCACCCAGCAGTCTGGCGATACCAAGGGGTGCATCCACTGCTCCTGCGACTCCCCCGACTGCGTCCACACTCTGATCAAGACCCTCTGCGGCCTCCGCGACCTCCTCCCCATGAACTAATCACCCCCTTATCCAGTGAAATAAAGATCATATTGATGATGATTTTACAGAAATAAAAAATAATCATTTGATTTGAAATAAAGATACAATCATATTGATGATTTGAGTTTAACAAAAAAATAAAGAATCACTTACTTGAAATCTGATACCAGGTCTCTGTCCATGTTTTCTGCCAACACCACTTCACTCCCCTCTTCCCAGCTCTGGTACTGCAGGCCCCGGCGGGCTGCAAACTTCCTCCACACGCTGAAGGGGATGTCAAATTCCTCCTGTCCCTCAATCTTCATTTTATCTTCTATCAGATGTCCAAAAAGCGCGTCCGGGTGGATGATGACTTCGACCCCGTCTACCCCTACGATGCAGACAACGCACCGACCGTGCCCTTCATCAACCCCCCCTTCGTCTCTTCAGATGGATTCCAAGAGAAGCCCCTGGGGGTGTTGTCCCTGCGACTGGCCGACCCCGTCACCACCAAGAACGGGGAAATCACCCTCAAGCTGGGAGAGGGGGTGGACCTCGATTCCTCGGGAAAACTCATCTCCAACACGGCCACCAAGGCCGCCGCCCCTCTCAGTTTTTCCAACAACACCATTTCCCTTAACATGGATCACCCCTTTTACACTAAAGATGGAAAATTATCCTTACAAGTTTCTCCACCATTAAATATACTGAGAACAAGCATTCTAAACACACTAGCTTTAGGTTTTGGATCAGGTTTAGGACTCCGTGGCTCTGCCTTGGCAGTACAGTTAGTCTCTCCACTTACATTTGATACTGATGGAAACATAAAGCTTACCTTAGACAGAGGTTTGCATGTTACAACAGGAGATGCAATTGAAAGCAACATAAGCTGGGCTAAAGGTTTAAAATTTGAAGATGGAGCCATAGCAACCAACATTGGAAATGGGTTAGAGTTTGGAAGCAGTAGTACAGAAACAGGTGTTGATGATGCTTACCCAATCCAAGTTAAACTTGGATCTGGCCTTAGCTTTGACAGTACAGGAGCCATAATGGCTGGTAACAAAGAAGACGATAAACTCACTTTGTGGACAACACCTGATCCATCACCAAACTGTCAAATACTCGCAGAAAATGATGCAAAACTAACACTTTGCTTGACTAAATGTGGTAGTCAAATACTGGCCACTGTGTCAGTCTTAGTTGTAGGAAGTGGAAACCTAAACCCCATTACTGGCACCGTAAGCAGTGCTCAGGTGTTTCTACGTTTTGATGCAAACGGTGTTCTTTTAACAGAACATTCTACACTAAAAAAATACTGGGGGTATAGGCAGGGAGATAGCATAGATGGCACTCCATATACCAATGCTGTAGGATTCATGCCCAATTTAAAAGCTTATCCAAAGTCACAAAGTTCTACTACTAAAAATAATATAGTAGGGCAAGTATACATGAATGGAGATGTTTCAAAACCTATGCTTCTCACTATAACCCTCAATGGTACTGATGACAGCAACAGTACATATTCAATGTCATTTTCATACACCTGGACTAATGGAAGCTATGTTGGAGCAACATTTGGGGCTAACTCTTATACCTTCTCATACATCGCCCAAGAATGAACACTGTATCCCACCCTGCATGCCAACCCTTCCCACCCCACTCTGTGGAACAAACTCTGAAACACAAAATAAAATAAAGTTCAAGTGTTTTATTGATTCAACAGTTTTACAGGATTCGAGCAGTTATTTTTCCTCCACCCTCCCAGGACATGGAATACACCACCCTCTCCCCCCGCACAGCCTTGAACATCTGAATGCCATTGGTGATGGACATGCTTTTGGTCTCCACGTTCCACACAGTTTCAGAGCGAGCCAGTCTCGGGTCGGTCAGGGAGATGAAACCCTCCGGGCACTCCCGCATCTGCACCTCACAGCTCAACAGCTGAGGATTGTCCTCGGTGGTCGGGATCACGGTTATCTGGAAGAAGCAGAAGAGCGGCGGTGGGAATCATAGTCCGCGAACGGGATCGGCCGGTGGTGTCGCATCAGGCCCCGCAGCAGTCGCTGCCGCCGCCGCTCCGTCAAGCTGCTGCTCAGGGGGTCCGGGTCCAGGGACTCCCTCAGCATGATGCCCACGGCCCTCAGCATCAGTCGTCTGGTGCGGCGGGCGCAGCAGCGCATGCGGATCTCGCTCAGGTCGCTGCAGTACGTGCAACACAGAACCACCAGGTTGTTCAACAGTCCATAGTTCAACACGCTCCAGCCGAAACTCATCGCGGGAAGGATGCTACCCACGTGGCCGTCGTACCAGATCCTCAGGTAAATCAAGTGGTGCCCCCTCCAGAACACGCTGCCCACGTACATGATCTCCTTGGGCATGTGGCGGTTCACCACCTCCCGGTACCACATCACCCTCTGGTTGAACATGCAGCCCCGGATGATCCTGCGGAACCACAGGGCCAGCACCGCCCCGCCCGCCATGCAGCGAAGAGACCCCGGGTCCCGGCAATGGCAATGGAGGACCCACCGCTCGTACCCGTGGATCATCTGGGAGCTGAACAAGTCTATGTTGGCACAGCACAGGCATATGCTCATGCATCTCTTCAGCACTCTCAACTCCTCGGGGGTCAAAACCATATCCCAGGGCACGGGGAACTCTTGCAGGACAGCGAACCCCGCAGAACAGGGCAATCCTCGCACAGAACTTACATTGTGCATGGACAGGGTATCGCAATCAGGCAGCACCGGGTGATCCTCCACCAGAGAAGCGCGGGTCTCGGTCTCCTCACAGCGTGGTAAGGGGGCCGGCCGATACGGGTGATGGCGGGACGCGGCTGATCGTGTTCGCGACCGTGTCATGATGCAGTTGCTTTCGGACATTTTCGTACTTGCTGTAGCAGAACCTGGTCCGGGCGCTGCACACCGATCGCCGGCGGCGGTCTCGGCGCTTGGAACGCTCGGTGTTGAAATTGTAAAACAGCCACTCTCTCAGACCGTGCAGCAGATCTAGGGCCTCAGGAGTGATGAAGATCCCATCATGCCTGATGGCTCTGATCACATCGACCACCGTGGAATGGGCCAGACCCAGCCAGATGATGCAATTTTGTTGGGTTTCGGTGACGGCGGGGGAGGGAAGAACAGGAAGAACCATGATTAACTTTTAATCCAAACGGTCTCGGAGTACTTCAAAATGAAGATCGCGGAGATGGCACCTCTCGCCCCCGCTGTGTTGGTGGAAAATAACAGCCAGGTCAAAGGTGATACGGTTCTCGAGATGTTCCACGGTGGCTTCCAGCAAAGCCTCCACGCGCACATCCAGAAACAAGACAATAGCGAAAGCGGGAGGGTTCTCTAATTCCTCAATCATCATGTTACACTCCTGCACCATCCCCAGATAATTTTCATTTTTCCAGCCTTGAATGATTCGAACTAGTTCCTGAGGTAAATCCAAGCCAGCCATGATAAAGAGCTCGCGCAGAGCGCCCTCCACCGGCATTCTTAAGCACACCCTCATAATTCCAAGATATTCTGCTCCTGGTTCACCTGCAGCAGATTGACAAGCGGAATATCAAAATCTCTGCCGCGATCCCTGAGCTCCTCCCTCAGCAATAACTGTAAGTACTCTTTCATATCCTCTCCGAAATTTTTAGCCATAGGACCACCAGGAATAAGATTAGGGCAAGCCACAGTACAGATAAACCGAAGTCCTCCCCAGTGAGCATTGCCAAATGCAAGACTGCTATAAGCATGCTGGCTAGACCCGGTGATATCTTCCAGATAACTGGACAGAAAATCGCCCAGGCAATTTTTAAGAAAATCAACAAAAGAAAAATCCTCCAGGTGGACGTTTAGAGCCTCGGGAACAACGATGAAGTAAATGCAAGCGGTGCGTTCCAGCATGGTTAGTTAGCTGATCTGTAGAAAAAACAAAAATGAACATTAAACCATGCTAGCCTGGCGAACAGGTGGGTAAATCGTTCTCTCCAGCACCAGGCAGGCCACGGGGTCTCCGGCGCGACCCTCGTAAAAATTGTCGCTATGATTGAAAACCATCACAGAGAGACGTTCCCGGTGGCCGGCGTGAATGATTCGACAAGATGAATACACCCCCGGAACATTGGCGTCCGCGAGTGAAAAAAAGCGCCCGAGGAAGCAATAAGGCACTACAATGCTCAGTCTCAAGTCCAGCAAAGCGATGCCATGCGGATGAAGCACAAAATTCTCAGGTGCGTACAAAATGTAATTACTCCCCTCCTGCACAGGCAGCAAAGCCCCCGATCCCTCCAGGTACACATACAAAGCCTCAGCGTCCATAGCTTACCGAGCAGCAGCACACAACAGGCGCAAGAGTCAGAGAAAGGCTGAGCTCTAACCTGTCCACCCGCTCTCTGCTCAATATATAGCCCAGATCTACACTGACGTAAAGGCCAAAGTCTAAAAATACCCGCCAAATAATCACACACGCCCAGCACACGCCCAGAAACCGGTGACACACTCAAAAAAATACGCGCACTTCCTCAAACGCCCAAAACTGCCGTCATTTCCGGGTTCCCACGCTACGTCATCAAAACACGACTTTCAAATTCCGTCGACCGTTAAAAACGTCACCCGCCCCGCCCCTAACGGTCGCCCGTCTCTCAGCCAATCAGCGCCCCGCATCCCCAAATTCAAACACCTCATTTGCATATTAACGCGCACAAAAAGTTTGAGGTATATTATTGATGATGG

SEQ ID NO: 8 - protein, 21 aa, Severe acute respiratory syndrome-related coronavirus 2
WYIWLGFIAGLIAIVMVTIML

SEQ ID NO: 9 - protein, 44 aa, Listeria monocytogenes
MDFSNMSILHYLANIVDILVVWFVIYKVIMLIRGTKAVQLLKGI

(19) United States
(12) Patent Application Publication
(10) Pub. No.: US 2025/0101066 A1
(43) Pub. Date: Mar. 27, 2025

(54) Viral Vaccines

(71) Applicants: McMaster University, Hamilton (CA); Ottawa Hospital Research Institute, Ottawa (CA); Turnstone Biologics Inc., Ottawa (CA)

(72) Inventors: Brian Lichty, Brantford (CA); Carolina Ilkow, Ottawa (CA); Zhou Xing, Ancaster (CA); Myrna Dolovich, Hamilton (CA); Fiona Smaill, Burlington (CA); Kyle Stephenson, Ottawa (CA)

(21) Appl. No.: 18/576,914

(22) PCT Filed: Jul. 15, 2022
(86) PCT No.: PCT/CA2022/051107

§ 371 Date: Jan. 5, 2024

Related U.S. Application Data
(60) Provisional application No. 63/222,723, filed on Jul. 16, 2021.

(51) Int. Cl. C07K 14/005; A61K 39/00; A61P 31/14; C12N 9/12; C12N 15/86

(52) U.S. Cl. CPC C07K 14/005; A61P 31/14; C12N 9/127; C12N 15/86; A61K 2039/53; A61K 2039/543; A61K 2039/70; C07K 2319/03; C12N 2710/10343; C12N 2760/20222; C12N 2760/20234; C12N 2770/20022; C12N 2770/20034; C12N 2770/32022; C12N 2770/32034

(57) ABSTRACT

A trivalent transgene that encodes a viral surface glycoprotein component, a viral nucleoprotein component and a viral RNA polymerase component is provided. Vaccines incorporating the trivalent transgene are also provided, along with methods of vaccinating mammals to protect against viral infection.

### FIELD OF THE INVENTION

The present invention generally relates to viral vaccines and methods of vaccination, and more particularly, relates to vaccines and methods of vaccinating against RNA viruses, such as positive-strand RNA viruses.

### BACKGROUND OF THE INVENTION

Positive-sense RNA (+ssRNA) viruses make up more than one-third of all known virus genera. Their genome is similar to an mRNA strand which can be directly translated to express proteins needed for transcription and replication. These types of viruses use host factors in all steps of viral infection, such as entry and replication. Perhaps more importantly, +ssRNA viruses can modulate the gene expression and defenses of the host by appropriating host factors.

Positive-sense RNA viruses include pathogens such as the Hepatitis C virus, West Nile virus, Dengue virus, and MERS, SARS, and SARS-CoV-2 coronaviruses, as well as less clinically serious pathogens such as the coronaviruses and rhinoviruses that cause the common cold.

Coronavirus Disease 2019 (COVID-19) is a classified human disease caused by Severe Acute Respiratory Syndrome Coronavirus 2 (SARS-CoV-2) that initially appeared in Wuhan, China in late December, 2019. Two months later, in March 2020, the World Health Organization (WHO) labelled the outbreak a pandemic. As of Jan. 10, 2021, 1,914,378 people died from the disease, out of a total of 88,120,981 confirmed cases reported to WHO globally. As these numbers continue to grow at a rapid pace, the international medical and scientific community are facing an unprecedented challenge to minimize the health and economic impacts of this disease.

Crucial to this effort is the provision of effective preventative treatments to minimize the spread of infection, and in particular, to reduce the incidence of severe disease and death from infection by SARS-CoV-2, variants thereof and related viral infection. A few vaccines have been developed and are currently in use. These include both mRNA and adenoviral-based vaccines that encode the SARS-CoV-2 spike protein. While these vaccines have been indicated to exhibit relatively high levels of efficacy, they are based on the production of the specific SARS-CoV-2 spike protein, and thus, their efficacy against SARS-CoV-2 variants is unclear, and efficacy against other related coronaviruses is doubtful.

Accordingly, it would be desirable to develop a novel broad spectrum vaccine for use generally against positive-sense RNA viruses.

### SUMMARY OF THE INVENTION

A novel trivalent vaccine has now been developed comprising a transgene that encodes three viral antigens, a surface glycoprotein component, a nucleoprotein component and an RNA polymerase component.

Thus, in one aspect of the invention, a trivalent transgene that encodes a viral surface glycoprotein component, a viral nucleoprotein component and a viral RNA polymerase component is provided, as well as a vaccine comprising the trivalent transgene, and methods of vaccinating a mammal using the vaccine.

In another aspect of the invention, a trivalent transgene is provides that encodes: i) a first viral surface glycoprotein component comprising a signal peptide and a viral surface glycoprotein coupled to a transmembrane domain that targets an extracellular vesicle and functions to anchor the surface glycoprotein to the vesicle for extracellular expression; and ii) a second protein comprising a viral nucleoprotein component that complexes or associates with viral RNA and a viral RNA polymerase component, wherein the first glycoprotein component and the second protein are linked with a self-cleaving peptide.

In another aspect of the invention, a coronaviral vaccine comprising a trivalent transgene is provided that encodes a coronaviral spike protein component, nucleoprotein component and RNA polymerase component.

In a further aspect of the invention, a method of vaccinating a mammal against infection by a virus such as a single-stranded RNA virus is provided, comprising treating the mammal with a vaccine comprising a trivalent transgene that encodes a surface glycoprotein component derived from the single-stranded RNA virus, a viral nucleoprotein component and a viral RNA polymerase component.

In further aspects of the invention, a replication-incompetent E1/E3-deleted adenoviral vector, a kit comprising an adenovirally-vectored vaccine in combination with a nebulizer, and a method of vaccinating a mammal against infection by a virus comprising administering to the mammal an adenovirally-vectored vaccine with a nebulizer are provided.

These and other embodiments of the invention are described in the detailed description and specific examples by reference to the following Figures.

### BRIEF DESCRIPTION OF THE FIGURES

FIG. 1 is a schematic of a transgene for incorporation into a vaccine in accordance with an embodiment of the invention;

FIG. 1B illustrates an amino acid sequence of an expressed transgene in accordance with an embodiment of the invention;

FIG. 2 illustrates A) the expression in human cells transduced with chimpanzee (ChAd) and human (huAd) adenoviral vectors bearing the transgene of FIG. 1; and B) the expression in human cells transduced at variable MOI with two different adenoviral vectors bearing the transgene of FIG. 1;

FIG. 3 illustrates A) a vaccination and SARS-CoV2 viral challenge protocol, and graphically illustrates B) weight loss and C) survival following SARS-CoV2 infection in treated and untreated mice;

FIG. 4 graphically illustrates A) production of antibodies to the spike component, including the full spike protein and the receptor-binding domain of spike (RBD) in mice vaccinated with vaccine comprising a transgene of FIG. 1; and B) illustrates serum neutralization of SARS-CoV2 in vitro using the vaccine;

FIG. 5 illustrates that vaccination with either A) ChAd or B) huAd bearing the transgene of FIG. 1 induced T cell responses to each of the antigen expressed by the transgene;

FIG. 6 illustrates amino acid sequences of 2A proteins;

FIG. 7 illustrates the experimental schema (A) and results (B-J) of treating mice infected with variant strains of SARS-CoV2 with a ChAd vector of FIG. 2;

FIG. 8 graphically illustrates time to nebulize to dryness at different fill volumes of 0.9N saline in the Aerogen Solo Micropump nebulizer. The time to completely aerosolize various fill volumes (0.2, 0.5, 1.0, 2.0 ml) of 0.9N saline was determined, operating the Aerogen Solo continuously on the bench with the Aerogen Control Unit. Three different Aerogen Solo nebulizers (A/B/C) were each tested, measuring the time to dryness with the 4 fluid volumes.

FIG. 9 graphically compares emitted dose (ED) between male and female human subjects. ED was determined by using an Aerogen Solo nebulizer with Control Unit and Salbutamol Sulphate as a tracer. Data shown are % Salbutamol recovered at mouth of 4 males and 4 females.

FIG. 10 graphically illustrates aerodynamic particle size distributions (APSD) of aerosol droplets of Salbutamol vs. Salbutamol with Vaccine. APSD was determined by using the Next Generation Impactor. Data are shown as the distribution of the mean amounts of salbutamol per NGI stage (as Control) and then with vaccine added. The amounts of salbutamol collected on each NGI stage were determined by UV spectroscopy.

FIG. 11 graphically illustrates viability of viral particles in aerosolized vaccine. Estimated amounts of viable AdHu5Ag85A vaccine present in aerosol droplets generated by Aeroneb® Micropump nebulizer were determined by using a viral plaque forming unit (PFU) assay. Data are expressed as average values of PFU in vaccine sample right before nebulization and in aerosol collected into buffer, representative of two independent experiments.

FIG. 12 is a schematic illustrating the construction of a recombinant replication-defective ChAd68 vector, including: A) fragments used for DNA assembly into the ChAd68 shuttle plasmid and genomic BAC. Matching overhangs of adjacent fragments are depicted by matching patterns; and B) linearized ChAd68 shuttle plasmid and genomic BAC for co-transfection into HEK293 cells. Homologous recombination at the ˜2.5 kb overlap between the 2 constructs generates the full-length ChAd68 vector with E1 being replaced by a transgene cassette and part of the E3 region being deleted.

FIG. 13 illustrates the DNA sequence of a chimpanzee adenovirus serotype 68 E1/E3 deleted genome with an HCMV promoter in accordance with an embodiment of the invention;

FIG. 14 illustrates: A) a schematic showing a vaccination protocol utilizing an adenoviral vaccine according to an embodiment of the invention; B) the levels of spike and RBD serum antibodies generated following vaccination; C) the results of a viral neutralization using the serum antibodies; D) level of RBD-specific B cells in the lung following vaccination; and E) the level of CD8+ T cells in the lung following vaccination.

FIG. 15 illustrates: A) a schematic showing a vaccination protocol utilizing an adenoviral vaccine according to an embodiment of the invention and subsequent infection with SARS-CoV-2 (Beta); B) % weight loss in treated vs control animals post infection; C) viral burden in the lungs post-infection; and D) viral burden in the brain post-infection;

FIG. 16 illustrates: A) a schematic showing a vaccination protocol utilizing an adenoviral vaccine according to an embodiment of the invention and subsequent infection with SARS-CoV-2 (Delta); B) % weight loss in treated vs control animals post infection; and C) viral burden in the lungs post-infection;

FIG. 17 illustrates: A) a schematic showing a vaccination protocol utilizing an adenoviral vaccine according to an embodiment of the invention and subsequent infection with SARS-CoV-2 (Omicron); B) % weight loss in treated vs control animals post infection; and C) viral burden in the lungs post-infection.

### DETAILED DESCRIPTION OF THE INVENTION

In one aspect, a vaccine comprising a trivalent transgene is provided. The transgene encodes a viral surface glycoprotein component, a viral nucleoprotein component and a viral RNA polymerase component. The vaccine is useful, in one embodiment, to protect against viral infection, including infection by RNA viruses such as positive single-stranded RNA viral infection.

A positive single-stranded (+ss) RNA virus is a virus comprising an RNA genome that corresponds with mRNA and may be directly translated to express viral proteins. These viruses are classified in three orders, namely, Nidovirales, Tymovirales and Picornavirales comprising about 30 viral families, notably including the Coronaviridae, Picornaviridae and the Flavivirudae viral families. All +ssRNA viruses encode a surface glycoprotein that plays a role in viral infectivity, a nucleoprotein the associates with the RNA genome of the virus, and an RNA-dependent RNA polymerase, among other proteins.

The transgene encodes a viral surface glycoprotein component that plays a role in viral infectivity, i.e. the surface glycoprotein binds to and fuses with a host cell to be infected. The surface glycoprotein component incorporated within the transgene is derived from a surface glycoprotein of the target virus, such as a +ssRNA virus, and will vary accordingly with the virus targeted by the vaccine. For example, for coronaviruses, the surface glycoprotein is the spike (S) glycoprotein. For the Hepatitis C virus, the surface glycoprotein is the E1 and/or E2 proteins. For Flaviviruses, the surface glycoprotein is the E protein. For inclusion into the present transgene, nucleic acid encoding the full-length surface glycoprotein of the target virus (a first target virus), or an immunogenic portion or fragment thereof, is incorporated. An immunogenic portion of the surface glycoprotein is a portion that provokes an immunogenic response, for example, a portion comprising an antigen. In one embodiment, the glycoprotein component encodes an extracellular portion of the glycoprotein, the receptor-binding domain thereof (e.g. the domain that binds to the host cell), an immunogenic fragment of either of these, or a combination of these regions in whole or in part.

As one of skill in the art will appreciate, the transgene, will also generally incorporate nucleic acid encoding an N-terminal signal or leader sequence of the glycoprotein component. The leader sequence generally comprises about 12-40 amino acids and, once translated, functions to translocate the glycoprotein for secretion. The signal peptide may be, e.g., the native signal peptide of the glycoprotein to be produced, a heterologous signal peptide, or a hybrid of the native and a heterologous signal peptide. Numerous signal peptides are used for production of secreted proteins, including but not limited to, murine immunoglobulin signal peptide (IgSP, EMBL Accession No. M13331), and leader sequences from other immunoglobulins, tissue plasminogen activator (tPA), insulin, Vesicular Stomatitis Virus glycoprotein (VSVG), IL-2, albumin, and chymotrypsin. Hybrid leader sequences have also been developed, for example, a leader sequence comprising an immunoglobulin signal peptide fused to a tissue-type plasminogen activator propeptide.

Nucleic acid encoding the surface glycoprotein component of the transgene is coupled to nucleic acid encoding a transmembrane (TM) domain that functions to traffic the glycoprotein for extracellular expression. The term “coupled” is used herein to convey that the surface glycoprotein is attached to the TM such that the TM effectively traffics the glycoprotein for extracellular expression. Thus, the TM may be any domain sufficient to traffic and anchor the glycoprotein for extracellular expression, while not having an adverse effect on host immune response. For example, the TM may be a TM native to the selected glycoprotein component, a heterologous transmembrane domain, or a functionally equivalent variant thereof.

Alternatively, the TM may be a peptide capable of tethering or anchoring the glycoprotein component to an extracellular vesicle (EV) such as an exosome, microvesicle, macrovesicle, oncosome, vesicle or the like, which is known to enhance the serological response and, thus, antibody induction. Such EV-directed transmembrane domains can originate from viruses (e.g. VSVG), or originate in cells (e.g. CD63 and Lamp2b). Membrane spanning domains may be single pass or may pass through the membrane multiple times, such as four times (quadruple pass, or tetraspanin). Likewise, by “EV-directed tetraspanin” is meant a subset of tetraspanins that are trafficked to EV membranes. Tetraspanins are a family of membrane proteins found in all 10 multicellular eukaryotes, and also referred to as the transmembrane 4 superfamily (TM4SF) proteins. They have four transmembrane alpha-helices and two extracellular domains, one short extracellular domain or loop, and one longer extracellular domain/loop. Although several protein families have four transmembrane alpha-helices, tetraspanins are defined by conserved amino acid sequences including four or more cysteine residues in the EC2 domain, with two in a highly 15 conserved ‘CCG’ motif. Examples of proteins that specifically direct to, and are enriched in, EV membranes including single pass and tetraspanin domains, are set out in Table 1.

TABLE 1
Examples of Proteins Comprising EV-directed Transmembrane Domains

| | Protein Name | | Single pass or | NCBI sequence ID for Glycoprotein |
|---|---|---|---|---|
| | (abbreviation) | Origin | Tetraspanin | or Genome or Sequence |
| D1m | CD63 (Murine) | Cell | Tetraspanin | MGI: 99529 |
| D1h | CD63 (Human) | Cell | Tetraspanin | NG_008347.1 |
| D2 | CD9 | Cell | Tetraspanin | NG_055677 |
| D3 | LAMP2B | Cell | Single | DQ895288.2 |
| D7 | VSVG-TM | Virus | Single | J02428.1 |
| D8 | CD81 | Cell | Tetraspanin | NG_023386.1 |
| D9 | CD82 | Cell | Tetraspanin | NG_023234.1 |
| D10 | LAMP1 | Cell | Single | NC_000013.11 |
| D11 | JUNIN virus | Virus | Single | NP_899218.1 |
| | glycoprotein TM | | | |
| D12 | LASSA fever virus | Virus | Single | AIT17400.1 |
| | glycoprotein TM | | | |
| D13 | LCMV glycoprotein | Virus | Single | AAX49341.1 |
| | TM | | | |
| D14 | SARS-CoV-2 | Virus | Single | WYIWLGFIAGLIAIVMVTIML |
| | glycoprotein TM | | | (SEQ ID NO: 8) |
| D15 | Tamiami virus | Virus | Single | AAN32955.1 |
| | glycoprotein TM | | | |
| | Domain | | | |
| D16 | Guanarito virus | Virus | Single | NP_899210.1 |
| | glycoprotein TM | | | |
| | Domain | | | |
| D17 | Machupo virus | Virus | Single | NP_899212.1 |
| | glycoprotein TM | | | |
| | Domain | | | |
| D18 | Sabia virus | Virus | Single | YP_089665 |
| | glycoprotein TM | | | |
| | Domain | | | |
| D19 | Parana virus | Virus | Single | AAN32957.1 |
| | glycoprotein TM | | | |
| | Domain | | | |
| D20 | CdaA TM Domain | Bacterial | Single | MDFSNMSILHYLANIVDILVVWFVIYKVIM |
| | | | | LIRGTKAVQLLKGI (SEQ ID NO: 9) |

The transgene also encodes a viral nucleoprotein component, i.e. a protein that complexes with or otherwise interacts or associates with the viral RNA. The nucleoprotein or nucleocapsid protein will vary with the virus targeted by the vaccine. For example, for coronaviruses, the nucleoprotein (N) comprises an N-terminal and a C-terminal RNA binding domain (NTD/CTD), separated by a central RNA-binding region, each of which are highly conserved. For the Hepatitis C virus and Flaviviruses, the nucleoprotein is the C (capsid) protein. For inclusion in the transgene, the nucleoprotein component may be derived from the same virus as the glycoprotein component, and thereby selected to target the same virus targeted by the glycoprotein component (the first target virus), or may be derived from a second different virus from that of the glycoprotein component, and thereby selected to target the second virus, including a variant of the first target virus, or a virus from the same or different viral family of the first target virus. The nucleoprotein component comprises nucleic acid encoding the selected full-length nucleoprotein, or an immunogenic fragment thereof, such as a region comprising human T cell epitopes.

The RNA polymerase component is derived from viral RNA-dependent RNA polymerase (RdRp) which is conserved by +ssRNA viruses, e.g. conserved within +ssRNA families. It is preferred, thus, to select as the RNA polymerase component, nucleic acid encoding a region of an RdRp that is conserved by +ssRNA viruses. Conserved regions are readily identified using techniques known in the art including sequence alignment from multiple isolates.

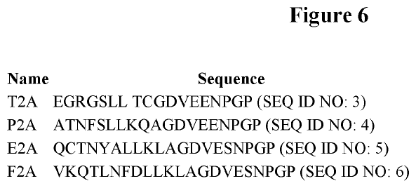

The surface glycoprotein component of the transgene may be linked to the nucleoprotein component of the transgene with a self cleaving peptide, for example, a peptide that induces ribosomal skipping thereby preventing formation of a peptide bond and resulting in expression of two discrete proteins from the single trivalent transgene. This facilitates the extracellular expression of the surface glycoprotein, while the nucleoprotein and polymerase components are expressed within the cell. Examples of self cleaving peptides include, but are not limited to, 2A peptides such as P2A (porcine teschovirus-1), E2A (equine rhinitis A virus), F2A (foot-and-mouth disease virus 18), and T2A (Thosea asigna virus 2A). The sequence of these self-cleaving peptides is shown in FIG. 6. As one of skill in the art will appreciate, sequences based on a 2A peptide sequence which vary from the native sequence but which retain the self-cleaving feature of the 2A peptide may also be used in the present transgene. The 2A peptide may include the optional linker “GSG” (Gly-Ser-Gly) on the N-terminal of a 2A peptide to enhance efficiency.

Among the +ssRNA viruses, the coronavirus is of significance. The coronavirus is an enveloped RNA virus with a nucleocapsid of helical symmetry and club-shaped spikes that project from surface. The coronaviral genome ranges in size from approximately 26 to 32 kilobases, and is one of the largest RNA viruses. Coronaviruses comprise four genera: Alphacoronavirus, Betacoronavirus, Gammacoronavirus and Deltacoronavirus, and of these, Alphacoronaviruses and Betacoronaviruses infect mammals. Coronaviruses (CoV) include common cold viruses, such as HCoV-229E, HCoV-HKU1, HCoV-NL63, and HCoV-OC43, and well as SARS-CoV. The term “SARS-CoV” refers to a coronavirus that causes severe acute respiratory syndrome. Examples include SARS-CoV1, SARS-CoV2 and MERS-CoV.

SARS-CoV-2 is an enveloped RNA virus of ˜30 kb comprising a 5′-UTR, two ORFs which encode 16 non-structural proteins such as RNA-dependent RNA polymerase, and a region that encodes structural proteins including a spike protein (S), envelope protein (E), membrane protein (M), and nucleocapsid protein (N). As used herein, SARS-CoV-2 is meant to encompass any SARS-CoV-2, including variant SARS-CoV-2 in which the genome sequence differs from the original SARS-CoV-2, including mutations in either or both coding and non-coding regions. In one embodiment, the SARS-CoV-2 has a genome sequence that essentially corresponds with the reference SARS-CoV-2 sequence (NCBI NC_045512), a 29903 nucleotide sequence in which the region spanning ribonucleotide positions 21563 . . . 25384 encodes the spike (S) protein, the region spanning 28274 . . . 29533 encodes the nucleoprotein (N), and the region spanning 266 . . . 21555 encodes the polymerase (POL).

The coronaviral spike protein includes a signal peptide located at the N-terminus, the S1 subunit, and the S2 subunit; the last two regions are responsible for receptor binding and membrane fusion, respectively. In the S1 subunit, there is an N-terminal domain and a receptor-binding domain (RBD); while the fusion peptide (FP), heptapeptide repeat sequence 1 (HR1), heptapeptide repeat sequence 2 (HR2), transmembrane (TM) domain, and cytoplasm domain comprise the S2 subunit.

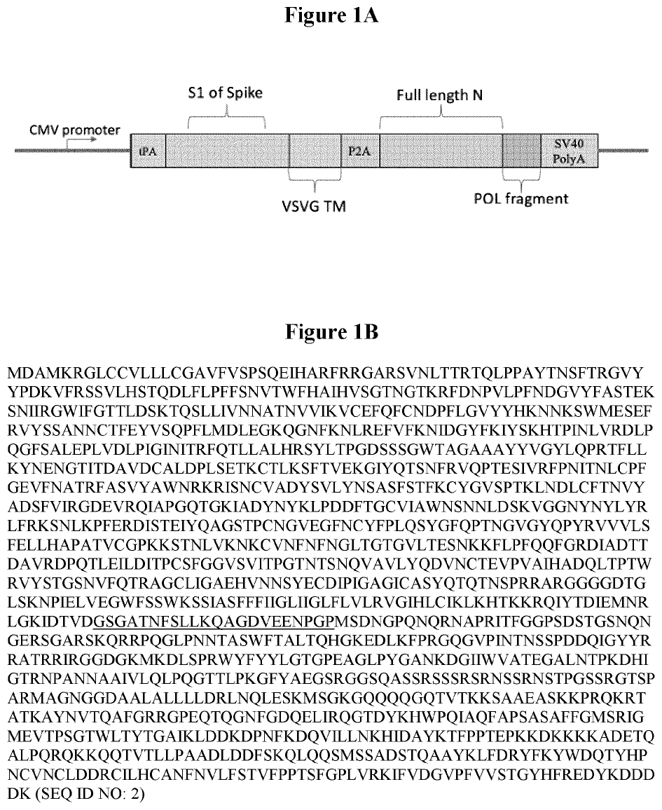

In one embodiment, the present transgene includes as the surface glycoprotein component nucleic acid encoding a coronaviral spike component selected from the full-length spike protein or an immunogenic portion thereof. For example, the spike component may comprise the full-length spike protein, the S1 subunit, or the RBD of S1 alone or with a portion of the N-terminal domain, or immunogenic fragments thereof. The spike component is selected based on the virus targeted by the vaccine. In one embodiment, the spike component is derived from a SARS-CoV, including SARS-CoV1, SARS-CoV2, or a variant of either of these, such as SARS-CoV2 Beta, Delta and Omicron variants. In one embodiment, the spike component is the S1 subunit having the amino acid sequence shown in FIG. 1B.

The spike protein component of the transgene is coupled to a transmembrane (TM) domain in order to traffic the spike protein for extracellular expression. The TM may be any domain sufficient to traffic and anchor the spike protein for extracellular expression, while not having an adverse effect on host immune response. For example, the TM may be the native coronavirus spike protein TM, or a functionally equivalent variant thereof. Alternatively, the TM may be a peptide capable of tethering or anchoring the spike component to an extracellular vesicle (EV) such as an exosome, microvesicle, macrovesicle, oncosome, vesicle or the like as previously described.

The coronavirus nucleoprotein component of the transgene may comprise nucleic acid encoding the full-length nucleoprotein, or an immunogenic fragment thereof, such as a region comprising human T cell epitopes which may be identified in silico or may be determined empirically by analyzing infected individuals. In one embodiment, the nucleoprotein component is that of a SARS-CoV, or a variant thereof. In one embodiment, the nucleoprotein component has the amino acid sequence shown in FIG. 1B.

The coronaviral RNA polymerase component of the transgene may comprise nucleic acid encoding the full-length polymerase, or an immunogenic fragment thereof such as a region comprising human T cell epitopes. In one embodiment, the RNA polymerase component is a region of the polymerase that is highly conserved among coronaviruses, and particularly bat-derived coronaviruses. The polymerase component may be derived from a SARS-CoV, such as SARS-CoV1 or SARS-CoV2, or a variant thereof. In one embodiment, the polymerase component has the amino acid sequence shown in FIG. 1B.

The spike component of the transgene is linked to the nucleoprotein component of the transgene with a self cleaving peptide, as previously described.

The transgene is constructed using well-established methods, and may be constructed for use in a nucleic acid-based vaccine, e.g. mRNA or DNA vaccine, or a viral-vectored vaccine.

For use in a DNA vaccine, the transgene coding regions for the glycoprotein component, such as a coronavirus spike protein component, nucleoprotein component and RNA polymerase component, may be synthesized, or otherwise obtained, for example commercially, and linked, as described, with DNA encoding the self-cleaving peptide, between the coding region for the spike protein component and the nucleoprotein/polymerase component. The vaccine construct will incorporate a suitable promoter to regulate expression of the transgene, as well as a transcriptional stop sequence, e.g. a poly (A) addition sequence, at the terminal end thereof. Examples of suitable promoters for incorporation in the vaccine construct, include but are not limited to, CMV, EF1a, CAG, PGK1, SV40, RSV, TRE, U6, UAS, Ubc, human beta actin, and CAG. The construct may also incorporate enhancer elements and/or transcriptional transactivators to enhance promoter activity when placed either upstream or downstream of the ORF.

The DNA transgene construct is then generally adapted for administration. The transgene construct may be formulated for administration as a linear molecule, covalently-closed linear construct or mini-circle. Alternatively, the transgene construct may be incorporated into a vector such as a plasmid or cosmid using techniques well-known in the art and then formulated for administration. The resulting DNA vaccine is formulated for administration. The vaccine may be incorporated within a delivery system adapted to enhance immunogenicity of the vaccine, for example, biodegradable polymeric microparticles (e.g. chitosan, polylacticecoglycolides, polyethyleneimine, amine-functionalized polymethacrylates, cationic poly(β-amino esters), poloxamers and polyvinylpyrrolidone polymers) or liposomes. The vaccine may also be combined with an adjuvant to enhance immunogenicity, e.g. inorganic compounds such as aluminum-containing compounds and squalene, oils such as paraffin, bacterial products such as toxoids, plant saponins, cytokines such as IL-1, IL-2 or IL-12, a cytosine phosphoguanine (CpG) motif-containing adjuvant, or an adjuvant combination such as Freund's adjuvant.

For use in an mRNA vaccine, the DNA transgene construct is prepared as described, and mRNA is synthesized therefrom by in vitro transcription of the cDNA template, typically plasmid DNA (pDNA), prepared as described using methods known in the art. Transcription of the cDNA template is conducted using RNA polymerase such as a bacteriophage RNA polymerase. For stability and efficient translation, the resultant mRNA strand will include a 5′ cap and 3′ poly(A) tail, as well as 5′ and 3′ untranslated regions (UTRs) flanking the coding region. The mRNA vaccine is then formulated for administration. In this regard, mRNA may be complexed with agents which prevent degradation, enhance uptake and promote translation. Examples of such adjuvants include, but are not limited to, cationic polypeptides (e.g. protamine), nanoemulsions, carrier peptides, liposomes, and immune activator proteins (e.g. CD70, CD40L, TLRs).

Viral-vectored vaccines may also be utilized to administer the present transgene construct, including both DNA viral vectors and RNA viral vectors.

DNA viral vector vaccines are adapted to expressibly incorporate the present DNA transgene construct, e.g. under the control of a viral promoter. Examples of suitable DNA viruses for use as vaccines include, but are not limited to, poxviruses such as vaccinia virus and modified vaccinia virus, adenoviruses, adeno-associated viruses, herpes simplex virus and cytomegalovirus, and including various serotypes thereof, both replication-competent and replication-deficient or replication-incompetent.

In one embodiment, a replication-incompetent adenovirus is prepared for use to deliver the present transgene construct. The transgene is incorporated within an E1/E3 region which has been deleted from the adenovirus.

RNA viral vector vaccines may also be adapted to expressibly incorporate an appropriate transcript of the present transgene construct, e.g. positive or negative strand. Examples of suitable RNA viruses for use as a vaccine to deliver the transgene include, but are not limited to, vesicular stomatitis viruses, retroviruses such as MoMLV, lentiviruses, Sendai viruses, measles-derived vaccines, Newcastle disease virus, alphaviruses such as Semliki Forest virus, flaviviruses, or an RNA replicon based on an RNA virus (i.e. derived from alphavirus, flavivirus, etc).

The present vaccine comprising a trivalent transgene as described in detail herein is used in a method of vaccinating a mammal against a viral infection, such as infection by an RNA virus such as a +ssRNA virus, e.g. a coronavirus. The vaccine is administered to the mammal in a therapeutically effective amount, i.e. an amount sufficient to generate in the mammal an immune response, generally a priming dose followed by a boosting dose. As one of skill in the art will appreciate, the amount required to generate an immune response will vary with a number of factors, including, for example, the particular transgene/antigens in the vaccine, the vector used to deliver the vaccine, and the mammal to be treated, e.g. species, age, size, etc. In this regard, for example, administration of a dosage in the range of about 10⁶ to 10⁸ pfu of adenoviral vector as a priming dose followed by about 10⁶ to 10⁸ pfu as a boosting dose, within about 2-16 weeks later, preferably, within about 4-12 weeks, to a mouse is sufficient to generate an immune response. A corresponding amount will generally be sufficient for administration to a human to generate an immune response.

The present vaccine is administered to a mammal to prevent a viral infection such as a coronaviral infection in any one of several administrable routes including, but not limited to, parenteral administration such as intravenously or intramuscularly, intranasally or by inhalation. For nucleic acid-based vaccines, other techniques such as administration by electroporation or using gene gun technology may be utilized. The prime and boosting vaccines, which may be the same or different vaccine type (e.g. both the prime and boosting vaccine may be a nucleic acid-based vaccine, or both may be a viral vectored vaccine, or the prime vaccine may be nucleic acid-based and the boosting vaccine may be a viral vector, or vice versa), may be administered by the same or different administrable routes. As will be appreciated by one of skill in the art, the vaccine, and any adjuvants, are administered in a suitable carrier, such as saline or other suitable buffer. The term “mammal” is used herein to refer to both human and non-human mammals.

In one embodiment, the present vaccine comprising a trivalent transgene as herein described is adapted to treat a +ssRNA respiratory infection, such as a coronaviral infection, is advantageously formulated for administration intranasally or by inhalation to directly target the respiratory mucosa. Preferably, the vaccine is provided as a viral-vectored vaccine in saline or other suitable buffer, to be nebulized to form a liquid aerosol for inhalation by mouth or via a nasal spray. In one embodiment, the vaccine is an adenoviral-vectored vaccine, e.g. huAd or ChAd. In another embodiment, the vaccine may be provided in kit form along with a nebulizer to assist with its administration.

Using this method, e.g. administration intranasally or by inhalation, the vaccine is efficiently delivered to the respiratory mucosa to generate immunological memory within the lungs, the target organ of a respiratory infection by a virus such as a coronavirus. This advantageously permits the use of a lower dose of vaccine to achieve effective protection. Furthermore, administration intranasally or by inhalation avoids vaccine administration using needles, which is perceived as undesirable by many, and also prevents generation of biohazardous sharps.

In another aspect of the invention, a method of delivering an adenoviral-vectored vaccine generally is provided comprising administration intranasally or by inhalation, for example, using a nebulizer. The method has been found to effectively deliver vaccine to the lungs using dosages which are significantly reduced in comparison to delivery by other routes, and avoids the disadvantages of other methods of administration.

The present vaccine comprising a trivalent transgene that expresses a viral surface glycoprotein, viral nucleoprotein and viral RNA dependent RNA polymerase component provides a broadspectrum vaccine. It advantageously results in generation of an immune response against a specific first target, e.g. an RNA virus such as an +ssRNA virus, e.g. a coronavirus, based on viral glycoprotein such as the spike protein that targets the first target virus, but is designed also to promote an immune response effective against a second target virus, either a related virus to the first target virus, such as a variant of the first target virus, or a different RNA virus (e.g. a +ssRNA virus) in view of the presence of the nucleoprotein and polymerase components. In this regard, it is noted that the nucleoprotein and polymerase components of SARS-CoV2 variants are highly conserved. Thus, a trivalent transgene in accordance with the invention which comprises a glycoprotein component that targets a SARS-CoV2 strain, as well as nucleoprotein and polymerase components that target the SARS-CoV2 strain, would be effective against not only the targeted strain, but also variants thereof. Thus, the vaccine is designed to provide protection against not only a target coronavirus, e.g. a SARS-CoV, but additionally provides protection against related coronaviruses, such as variants, that may emerge over time.

Embodiments of the invention are described by reference to the following specific examples which are not to be construed as limiting.

### EXAMPLES

### Example 1—Trivalent Transgene

A trivalent transgene, as shown in FIG. 1A, was prepared using established methods. The transgene comprised a human tPA signal sequence/propeptide (aa 1-32), the S1 region of SARS-CoV-2 spike protein (aa 47-716), the transmembrane domain of VSV G (aa 443-511) followed by the self-cleaving peptide, P2A of porcine teschovirus, having the sequence: GSGATNFSLLKQAGDVEENPGP (SEQ ID NO: 1), full-length SARS-CoV-2 nucleoprotein (aa 1-419) fused to a highly conserved portion of the SARS-CoV-2 polymerase (aa 4673-4742). The expression cassette gave rise to an approximately 86 kDa membrane bound Spike S1 antigen as well as an approximately 56 kDa intracellular nucleoprotein/POL fusion protein, the amino acid sequence of which is shown in FIG. 1B.

The transgene was incorporated into replication-incompetent human and chimpanzee adenoviral vectors, huAd5 and ChAd, using protocols well-established in the art.

### Example 2—Preparation of Replication-Incompetent ChAd Vector

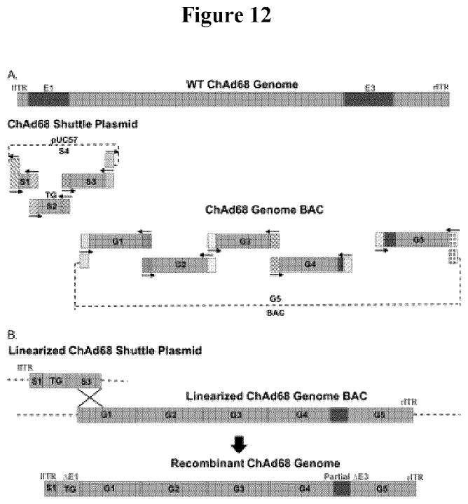

The replication-incompetent Chimpanzee Adenovirus 68 (ChAd68) vector was created by a two plasmid system based on the approach previously developed for other adenovirus vectors as described in Abbink et al. (Journal of virology, March 2018, 92(6), e01924-17). The shuttle plasmid contains the left inverted terminal repeat (ITR) through the pIX and pIVa2 sequences in which E1 is deleted and replaced by a transgene expression cassette. This cassette comprises of a human cytomegalovirus (HCMV) promoter, restriction enzyme sites for the convenient swapping of transgene coding sequences, and a simian virus 40 (SV40) polyadenylation signal. The remainder of the genome spanning from upstream of pIX to the right ITR with partial deletion of E3 was cloned into a bacterial artificial chromosome (BAC). The approximate 2.5 kb overlapping region between the shuttle plasmid and the genome BAC enables homologous recombination to generate the entire ChAd68 genome with a transgene cassette in place of E1 and partial E3 deletion when the 2 constructs are co-transfected into an E1-complementing cell line such as HEK293. To facilitate rapid cloning of the shuttle plasmid and the genome BAC, DNA assembly was utilized as described in Abbink et al. (2018). This method enables the joining of multiple DNA fragments that contain ˜20-60 nucleotide overlaps with their adjacent fragments in a single reaction without the requirement of restriction digest. FIG. 12 is a schematic illustration of the method used to prepare the ChAd vector.

To construct the shuttle plasmid using DNA assembly, four DNA fragments were generated by PCR in which the primers were designed to provide the required overlapping sequences between adjacent fragments. Phusion hot start II high fidelity DNA polymerase (Thermo Fisher) was used to reduce the risk of unintended mutations. The plasmid backbone fragment was obtained from a pUC57-based plasmid. The fragment containing an enhanced green fluorescent protein (eGFP) transgene cassette instead of E1 was PCR amplified using a synthetic DNA template. The other 2 fragments were amplified from genomic DNA extracted from wildtype ChAd68 (ATCC VR-594™). The PCR products were extracted from agarose electrophoresis gels and assembled into the shuttle vector by NEBuilder HiFi DNA assembly 2× master mix (New England BioLab) which allows for efficient seamless error-free ligation of the fragments. The assembled construct was then transformed into NEB 10-beta chemically competent Escherichia coli (New England BioLab), and colonies were sequentially screened for successful assembly by restriction digest and PCR at the junctions. Finally, the selected clones were verified to be free of undesirable mutations by Sanger sequencing.

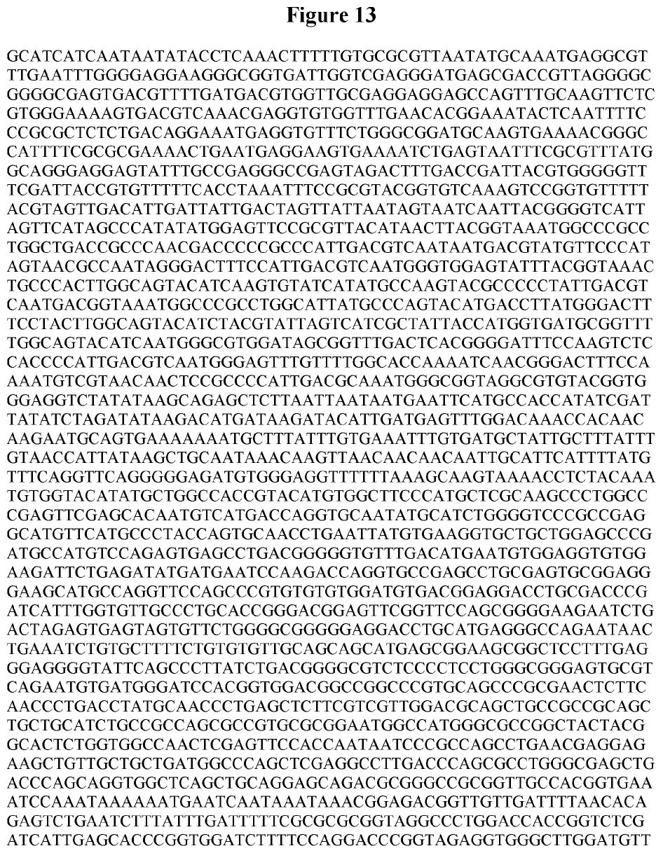

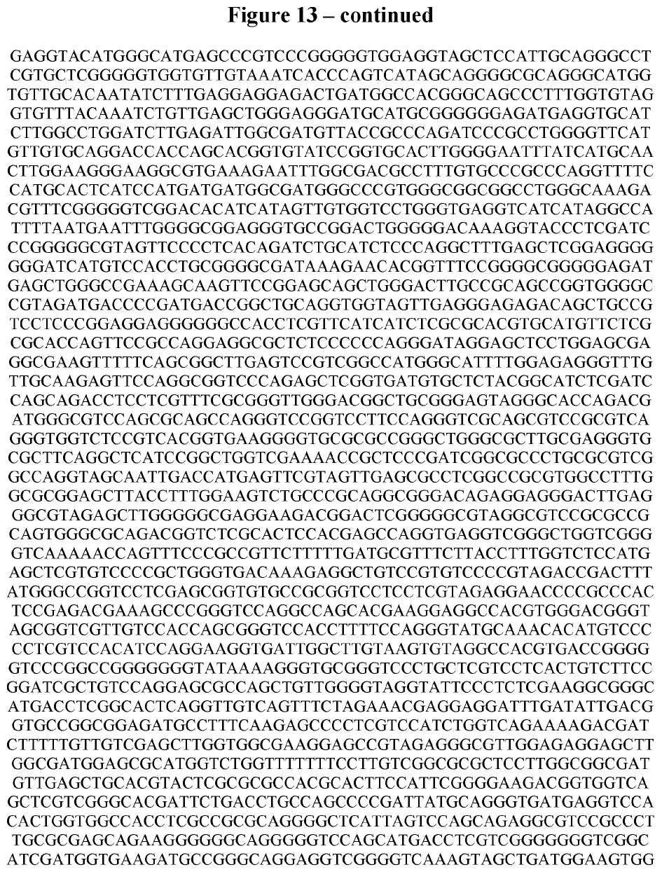

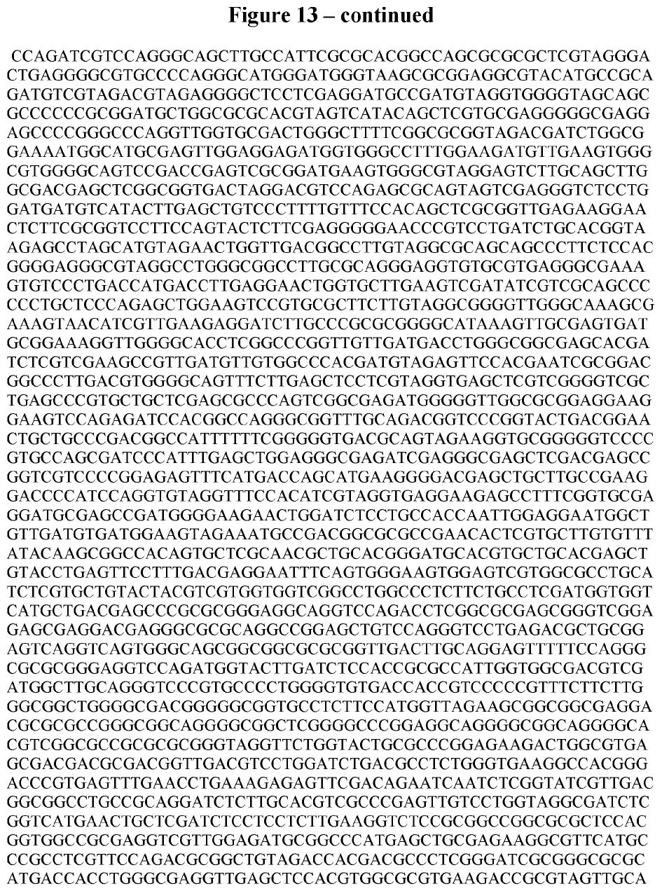

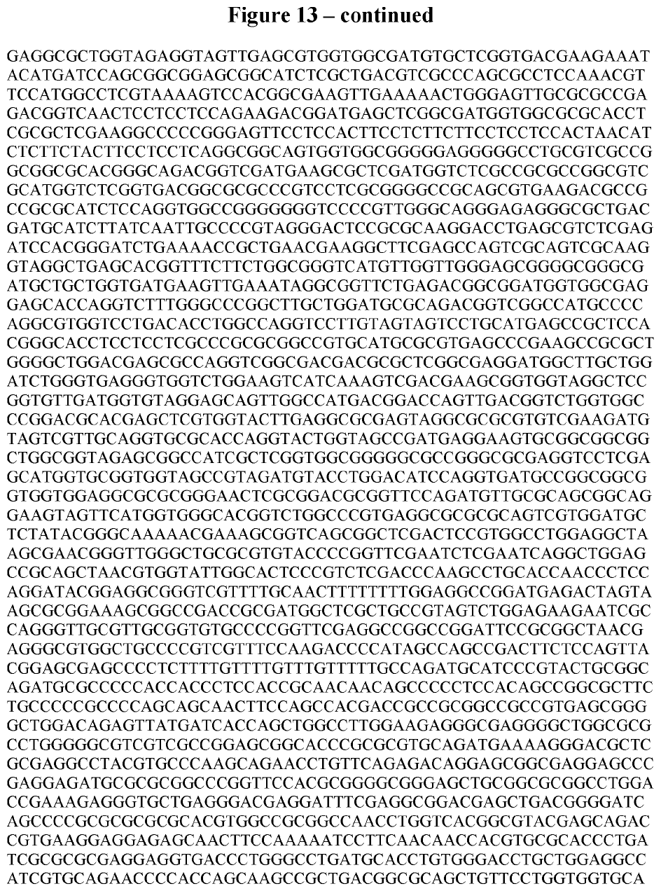

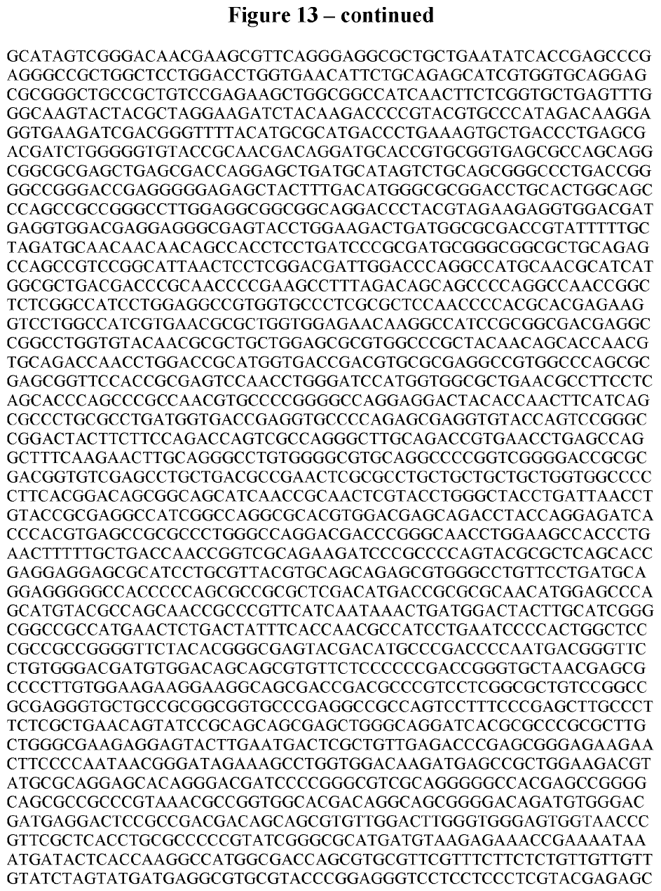

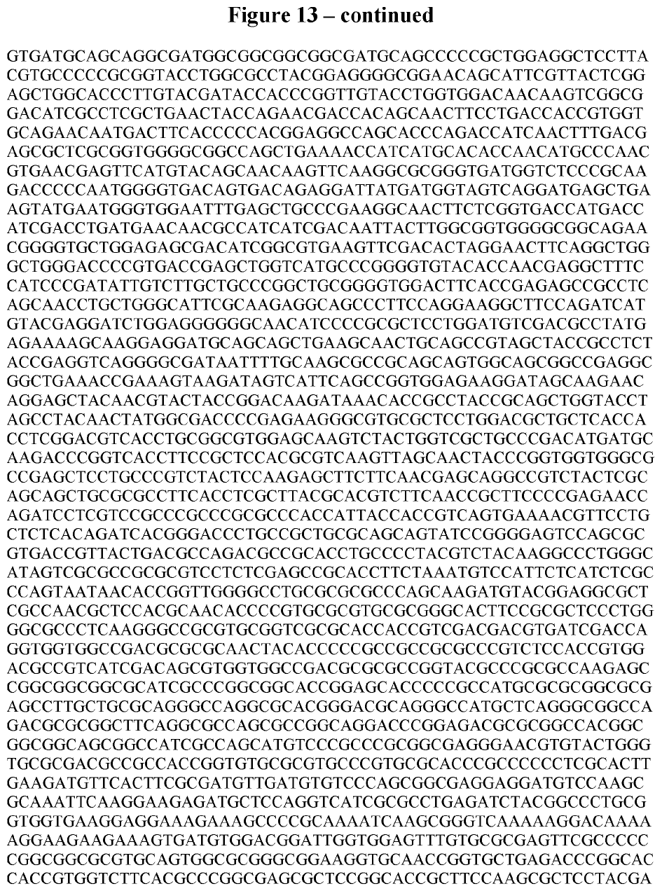

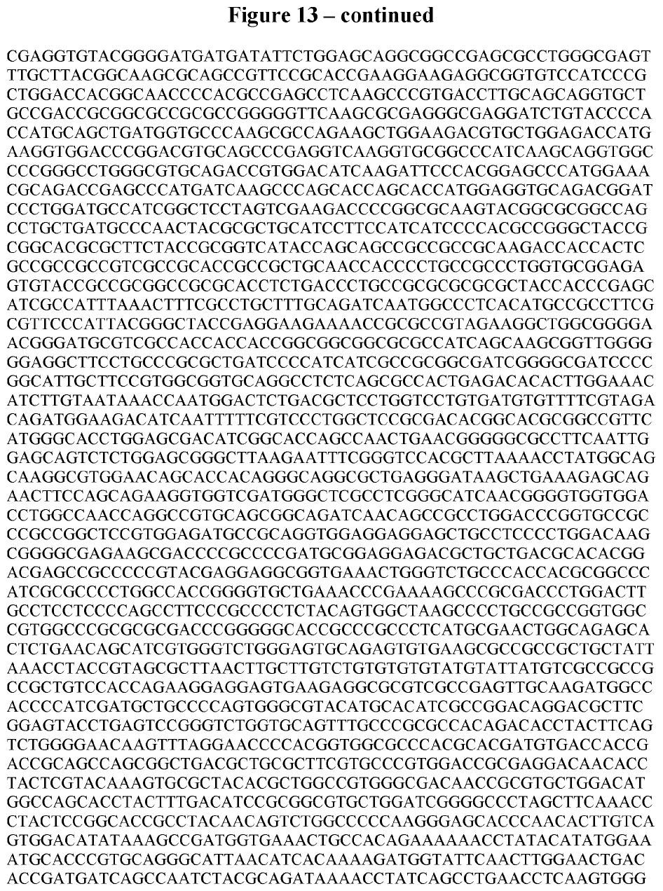

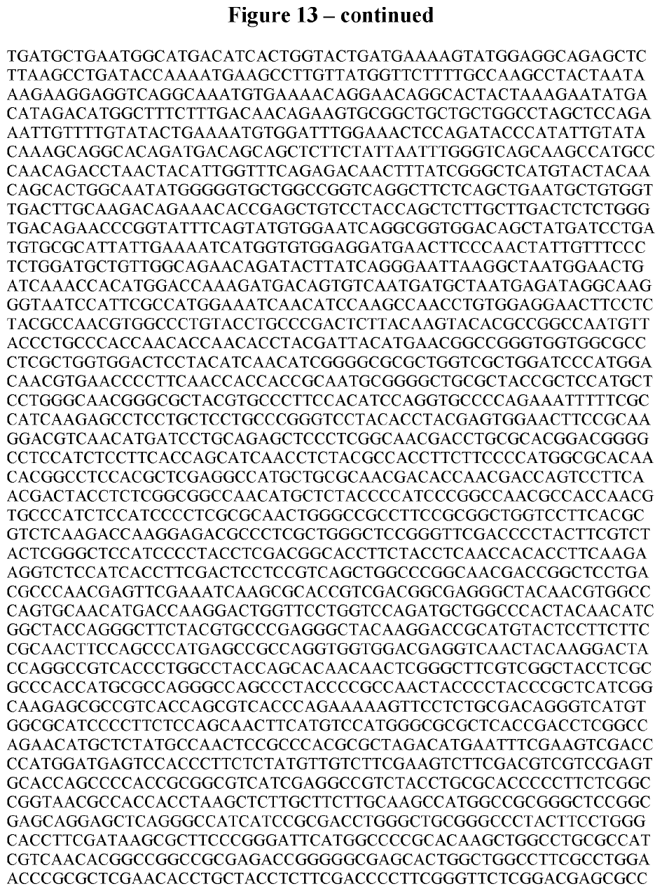

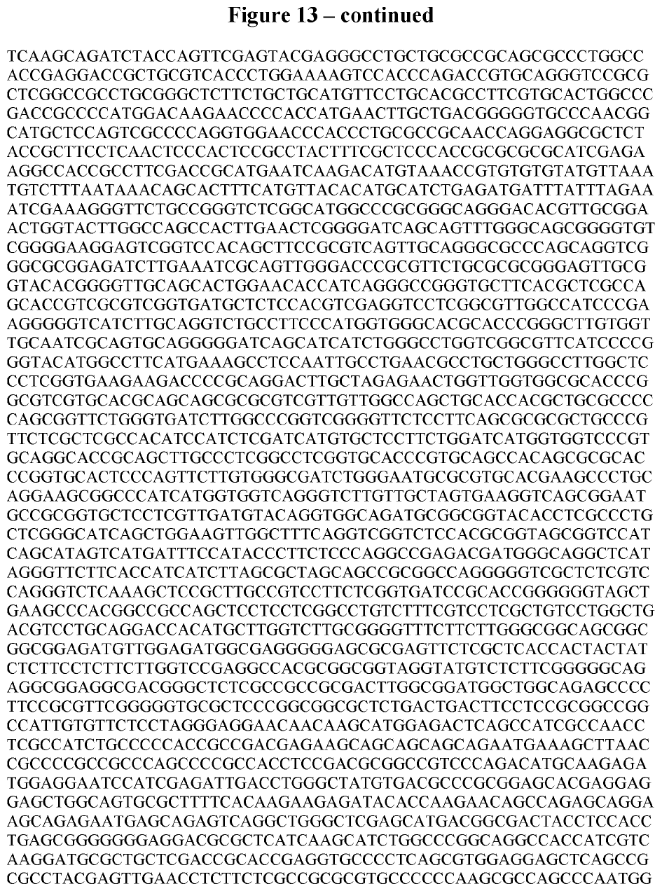

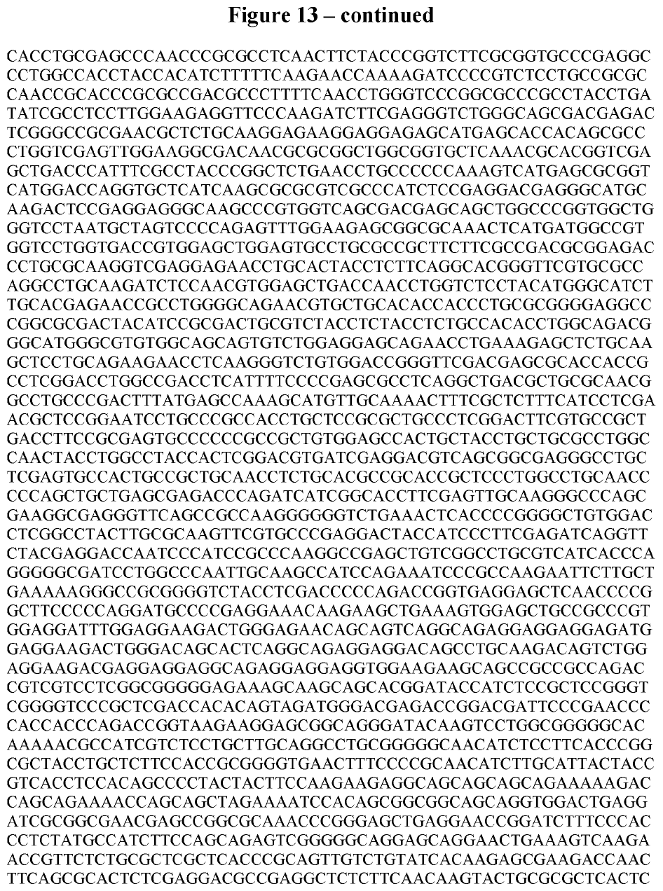

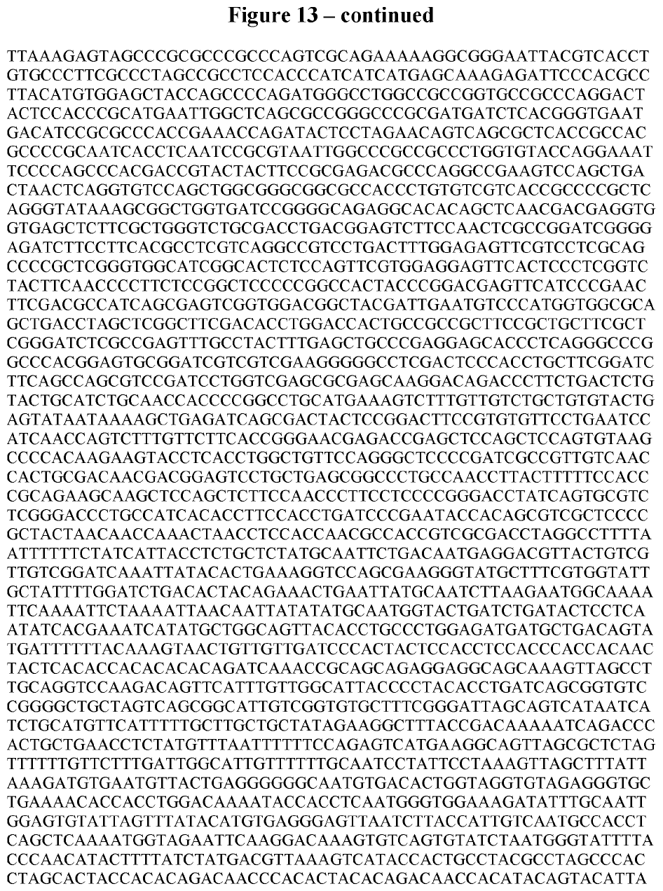

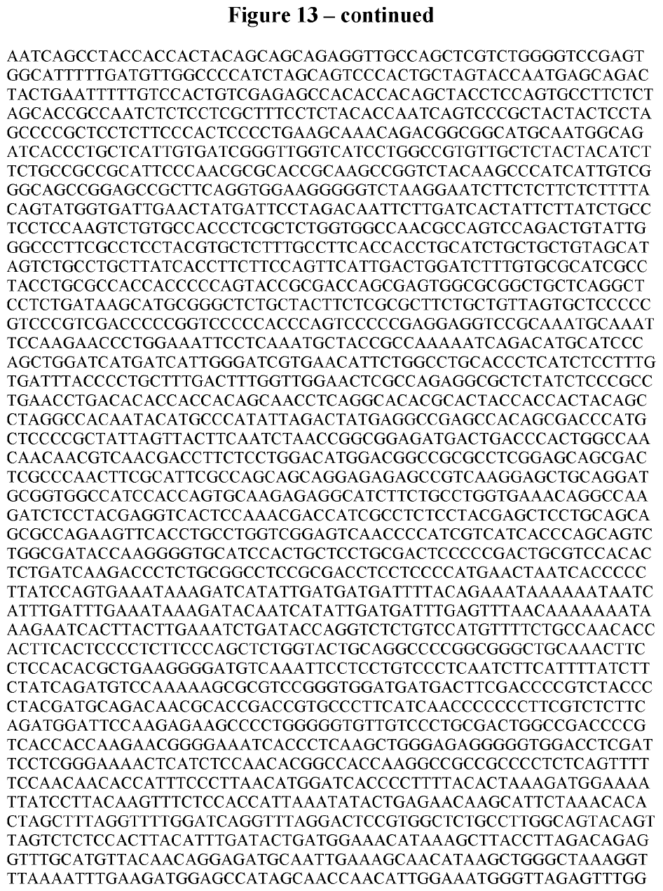

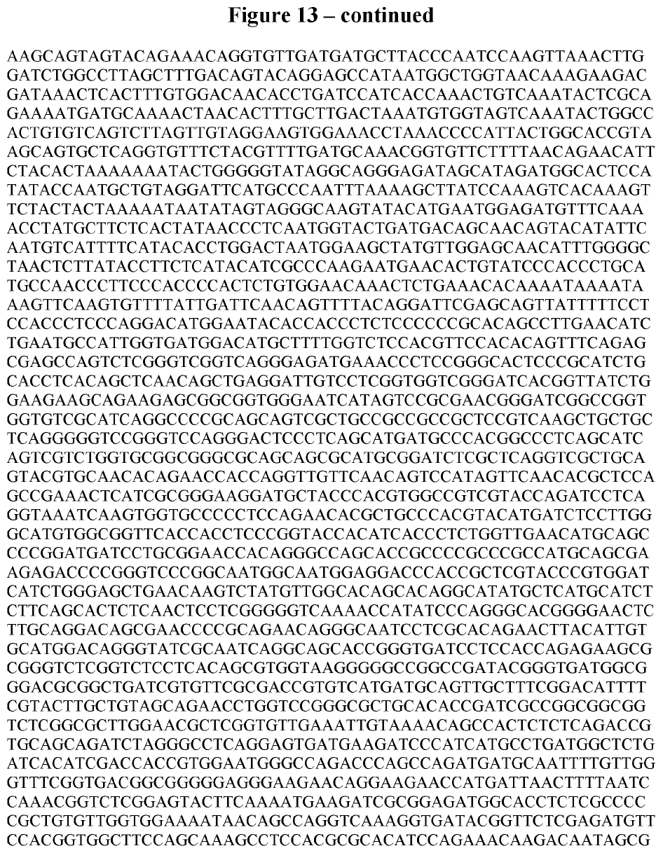

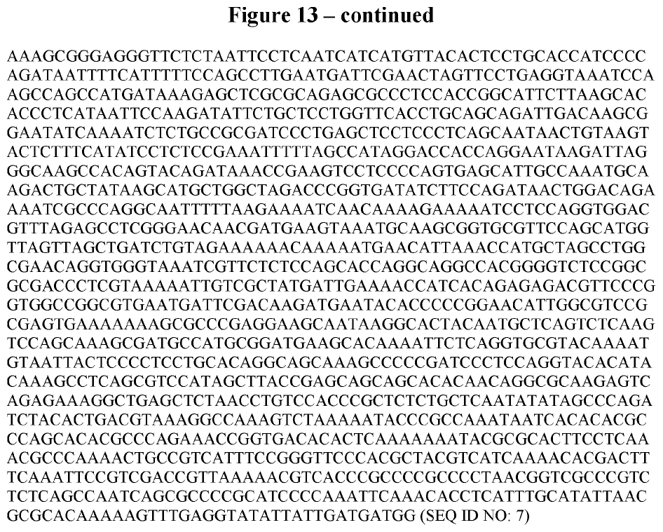

The genome BAC was built in a similar manner using NEBuilder HiFi DNA assembly with some modifications to accommodate the larger insert. The assembly reaction consists of a BAC backbone fragment prepared by restriction enzyme digestion and five ˜5.3-7.5 kb PCR fragments amplified from ChAd68 genomic DNA. A deletion of 1,388 nucleotides in the E3 region that disrupts 4 coding sequences (CR1-alpha1, gk19k, CR1-beta1, and CR1-gamma1) was achieved by joining 2 genomic fragments immediately flanking the sequence to be deleted. The assembled product was transformed into TransforMax EPI300 electrocompetent E. coli (Lucigen). Following colony selection by restriction digest and PCR, the sequences of the selected clones were confirmed by next generation sequencing. The sequence of adenoviral vector, ChAd68, is provided in FIG. 13.

The replication-incompetent huAd5 vector was similarly prepared.

A transgene, such as the transgene described in Example 1 (i.e. the trivalent SARS-CoV2 transgene), was readily subcloned into the ChAd68 shuttle plasmid using PacI or EcoRI and ClaI or XbaI sites at the 5′ and 3′ ends of the coding sequence, respectively. The shuttle plasmid containing the gene of interest and genome BAC were then linearized by restriction digest within the backbones, purified and co-transfected into HEK293 cells where they recombine to produce the desired ChAd68 vector comprising the transgene.

### Example 3

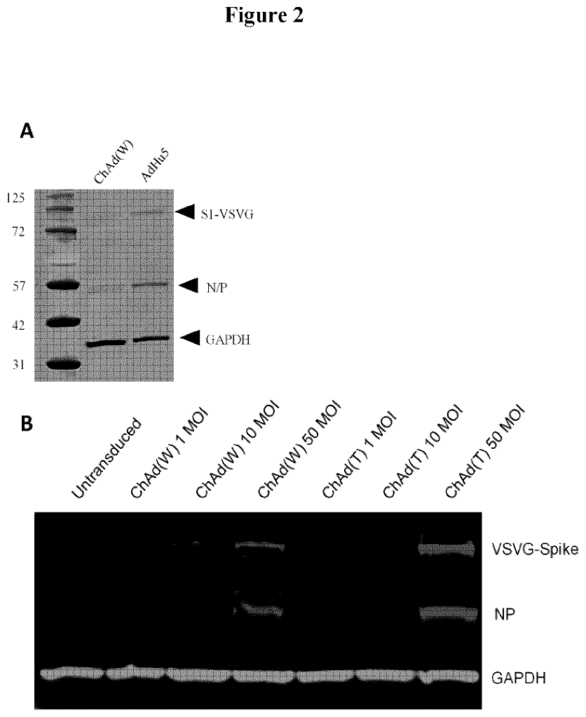

Human A549 cells were transduced with either replication-incompetent ChAd or huAd5 vaccine vectors bearing the trivalent SARS-CoV2 transgene, whole cell lysates were collected and Western blotted. Antibodies specific for the S1/VSVG fusion, the N/P fusion or cellular GAPDH were used to probe the blot. Discrete S1/VSVG and NP fusion proteins were expressed by transduced cells as shown in FIG. 2A.

Human A549 cells were transduced at various MOI with two different replication-incompetent ChAd clones (W or T) both bearing the trivalent SARS-CoV2 transgene, whole cell lysates were collected and Western blotted. Antibodies specific for the S1/VSVG fusion, the N/P fusion or cellular GAPDH were used to probe the blot. Discrete S1/VSVG and NP fusion proteins were expressed by transduced cells as shown in FIG. 2B.

### Example 4

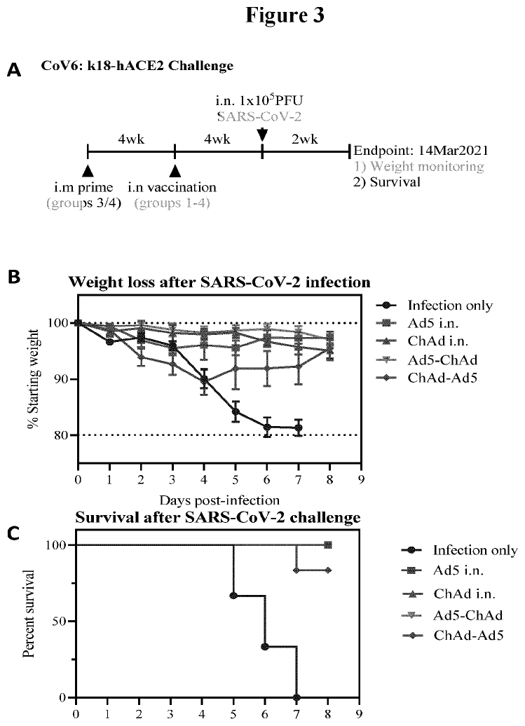

BALB/c mice were vaccinated using either a ChAd or huAd5 vaccine vector, or both in a prime-boost treatment (Ad5-ChAd, or ChAd-Ad5), expressing the trivalent SARS-CoV2 transgene using the protocol shown in FIG. 3A followed by viral challenge with SARS-CoV-2. The dosage of the vaccine vector administered was 5e7 PFU.

All treated groups maintained at least 95% of their starting weight over an 8 day period post-infection, with the exception of the ChAd-Ad5 treated group whose weight dipped to about 90% of starting weight at about 4 days post-infection (FIG. 3B).

As shown in FIG. 3C, survival was 100% for the Ad5 treated group, and somewhat less at about 80% in the ChAd-Ad5 group.

### Example 5

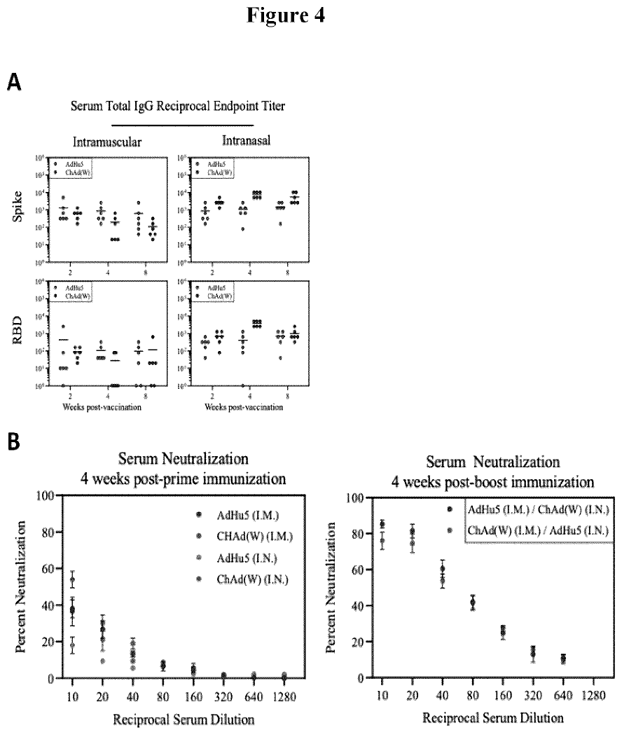

BALB/c mice were vaccinated as indicated using either a ChAd vaccine vector expressing the trivalent SARS-CoV2 transgene or a huAd5 vaccine vector expressing the trivalent SARS-CoV2 transgene. Spike- or RBD-specific antibodies were detected by ELISA at 2, 4 and 8 wks post-vaccination (FIG. 4A). Serum neutralization of SARS-CoV2 virus was performed in vitro. Results are shown in FIG. 4B.

### Example 6

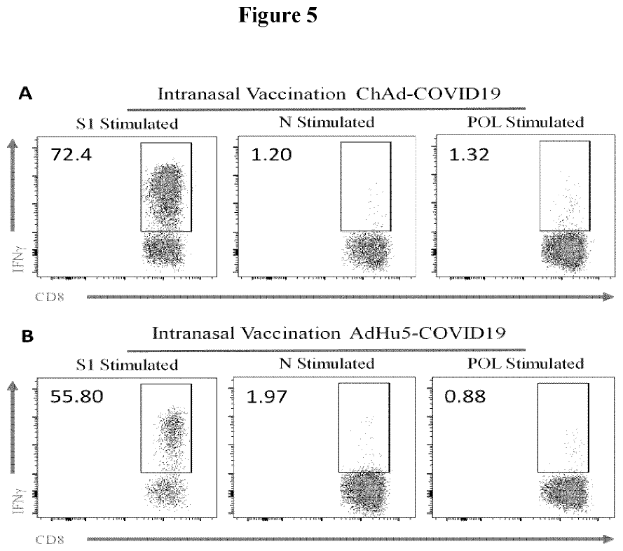

BALB/c mice were vaccinated by intranasal (inhalation) route using either a ChAd vaccine vector expressing the trivalent SARS-CoV2 transgene (Tri:ChAd) or a huAd5 vaccine vector expressing the trivalent SARS-CoV2 transgene. Four weeks later immune cells were collected from lungs by bronchoalveolar lavage (BAL) and assessed for specific T cell responses by stimulation with overlapping peptide libraries spanning the three SARS-CoV2 Ags; S1, N and POL. Both vectors induced CD8+ T cells specific for each of the three antigens in the lungs of vaccinated mice as shown in FIG. 5A/B.

### Example 7—Effect of Vaccination with Trivalent Transgene Against SARS-CoV2 Variants

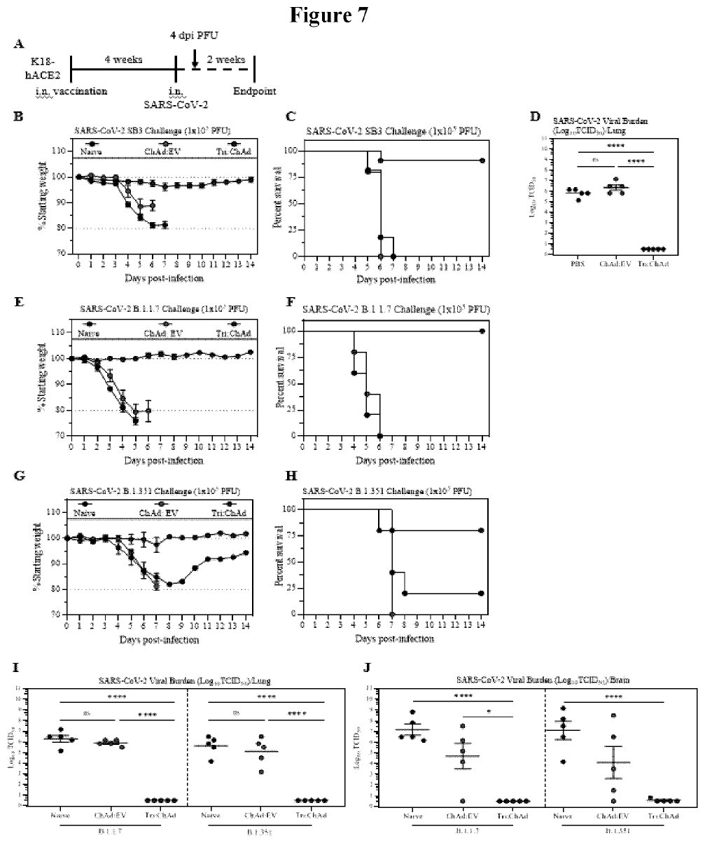

Animals (K18-hACE2 transgene mice, N=10 per group) were intranasally immunized with a single dose of 1×10⁷PFU Tri:ChAd or an empty vector (ChAd:EV) equivalent. Four weeks post-immunization, animals were intranasally infected with 1×10⁵ PFU of either SARS-CoV-2 ancestral (Wuhan) strain, B.1.1.7, or B.1.351. A cohort of animals was sacrificed (N=5) 4 days post-infection for lung and brain viral titers (see FIG. 7A). The remaining mice were monitored for weight loss and survival.

FIG. 7 illustrates weight loss (B) and survival (C) following intranasal infection with SARS-CoV-2 ancestral strain. Animals were monitored until a humane endpoint was reached (20% weight loss or neurological sequala), or until the experimental endpoint (14 days post-infection). As shown, vaccination with the transgene vector was effective against weight loss and sustain survival for the duration of the experiment. Lung viral titers determined at 4 days post-infection by performing plaque assays using whole lung homogenates showed essentially no viral burden in animals vaccinated with the transgene.

Weight loss and survival of vaccinated mice following infection with SARS-CoV2 variant B.1.1.7 and SARS-CoV2 variant B.1.351 were also monitored. Similar results were obtained (see FIGS. 7 E/G for weight loss data, and FIGS. 7 F/H for survival data) indicating that vaccination with the trivalent transgene was also effective against SARS-CoV2 variants. Lung viral titers in B.1.1.7 and B.1.351 infected animals were determined by performing plaque assays using whole lung homogenates (FIG. 7I). Brain viral titers B.1.1.7 and B.1.351 infected animals were determined by performing plaque assays using brain homogenates (FIG. 7J). Both show essentially no viral burden in vaccinated animals.

### Example 8

Six-to-eight week old female BALB/c mice were either intramuscularly (i.m.) or intranasally (i.n.) immunized with a single dose of Tri:ChAd (1×10⁷ PFU) and were subsequently sacrificed 8 weeks post-immunization.

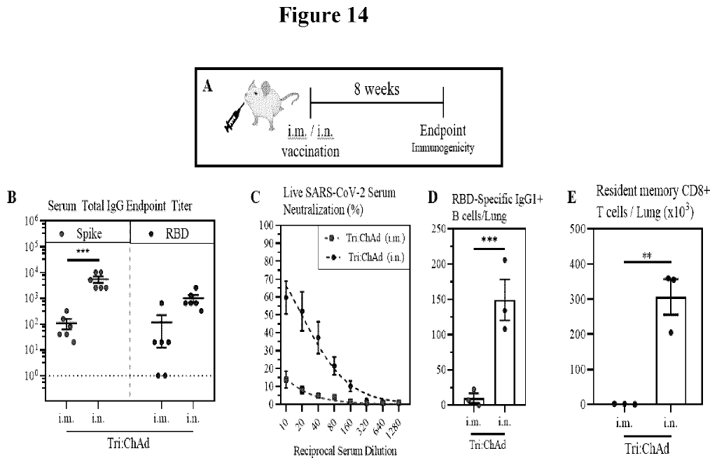

Serum from i.m. or i.n. immunized animals was utilized to assess for the presence of anti-Spike or anti-RBD binding antibodies by ELISA. Antibody levels are presented as endpoint titers. Spike and RBD proteins were synthesized based on the Wuhan-Hu-1 strain of SARS-CoV-2 (n=5 animals per group). As shown in FIG. 14B, respiratory mucosal vaccination (i.n.) generates increased levels of serum antibodies.

Serum neutralizing antibodies from i.m. or i.n. immunized animals against Wuhan-Hu-1 strain of SARS-CoV-2 were then used in a VERO-E6 based live virus neutralizing assay. Neutralization is presented as % neutralization relative to either uninfected or infected control wells (n=6 animals per group). As shown in FIG. 14C, antibodies from i.n immunized animals exhibited more effective neutralization in view of the increased level of antibody generated.

The absolute number of RBD-specific class-switched IgG1+ B cells were quantified from the lungs of either i.m. or i.n. immunized animals utilizing an RBD tetramer, by flow cytometry (n=3 animals per group). The absolute number of resident-memory CD8+ T cells (defined by co-expression of CD44, CD103, CD69, CD49a) were also quantified from the lungs of either i.m. or i.n. immunized animals, by flow cytometry (n=3 animals per group). As shown in FIG. 14D/E, respectively, the levels of both B and T cells in the lung of i.n. immunized animals was greater than that of i.m. immunized animals.

Thus, respiratory mucosal vaccination elicits robust and sustained adaptive immunity.

### Example 9

Six-to-eight week old female hemizygous K18-hACE2 mice (6.Cg-Tg(K18-ACE2)2Prlmn/J, Strain #: 034860, RRID:IMSR_JAX: 034860, Jackson Labs) were intranasally (i.n.) immunized with a single dose of Tri:ChAd (1×10⁷ PFU) and were subsequently i.n. infected with a lethal level of the SARS-CoV-2 Beta B.1.351 variant (1×10⁵ PFU). A subset of animals were sacrificed 4 days post-infection for enumeration of lung and brain viral titers. A remaining subset of animals were monitored for weight loss for 14 days total.

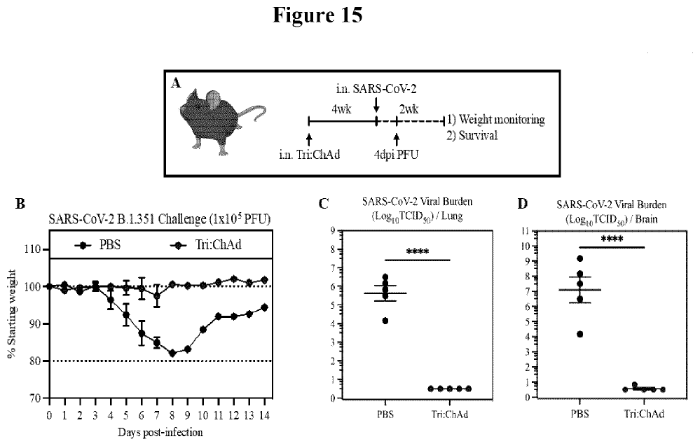

Weight loss was determined following i.n. infection with SARS-CoV-2 Beta B.1.351 variant in control (PBS) and i.n. Tri:ChAd-vaccinated animals (n=5 animals per group). As shown in FIG. 15B, the vaccinated animals exhibited little or no weight loss as compared to unvaccinated control animals.

Lung and brain viral titer enumeration 4 days post-infection with SARS-CoV-2 Beta B.1.351 variant in control (PBS) or i.n. Tri:ChAd-vaccinated animals was determined utilizing a VERO-E6 based assay and assessing for the presence of cytopathic effect (n=5 animals per group). As shown in FIGS. 15C/D, little or no viral burden was detectable in the lung and brain of vaccinated animals.

Thus, an adenoviral trivalent transgene such as Tri:ChAd is effective to protect against lethal infection with an immune evasive SARS-CoV-2 variant (beta variant).

### Example 10

Six-to-eight week old female hemizygous K18-hACE2 mice were intranasally (i.n.) immunized with a single dose of Tri:ChAd (1×10⁷ PFU) and were subsequently i.n. infected with SARS-CoV-2 Delta B.1.672 variant (1×10⁵ PFU). A subset of animals were sacrificed 4 days post-infection for enumeration of lung and brain viral titers. A remaining subset of animals were monitored for weight loss for 14 days total.

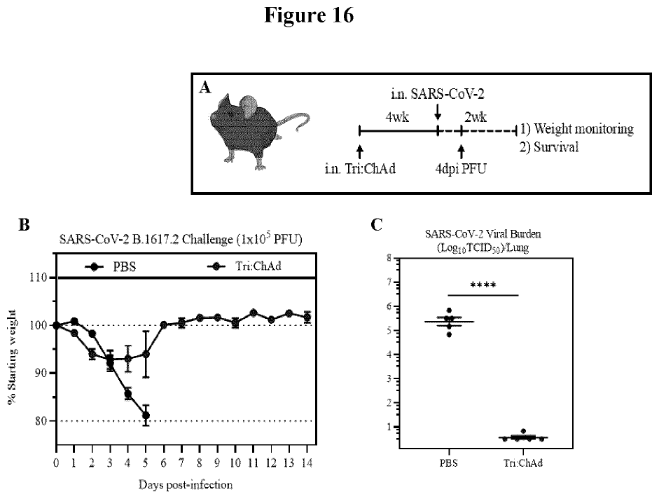

Weight loss was determined following i.n. infection with SARS-CoV-2 Delta B.1.672 in control (PBS) and i.n. Tri:ChAd-vaccinated animals (n=5 animals per group). As shown in FIG. 16B, the vaccinated animals exhibited much less weight loss as compared to unvaccinated control animals, and regained normal weights within about 5-6 days post-infection.

Lung viral titer enumeration 4 days post-infection with SARS-CoV-2 Delta B.1.672 in control (PBS) or i.n. Tri:ChAd-vaccinated animals was determined utilizing a VERO-E6 based assay and assessing for the presence of cytopathic effect (n=5 animals per group). As shown in FIG. 16C, little or no viral burden was detectable in the lung of vaccinated animals.

Thus, an adenoviral trivalent transgene such as Tri:ChAd is effective to protect against lethal infection with an immune evasive SARS-CoV-2 variant (delta variant).

### Example 11

Six-to-eight week old female hemizygous K18-hACE2 mice were intranasally (i.n.) immunized with a single dose of Tri:ChAd (1×10⁷ PFU) and were subsequently i.n. infected with SARS-CoV-2 Omicron BA.1 variant (1×10⁵ PFU). A subset of animals were sacrificed 4 days post-infection for enumeration of lung and brain viral titers. A remaining subset of animals were monitored for weight loss for 14 days total.

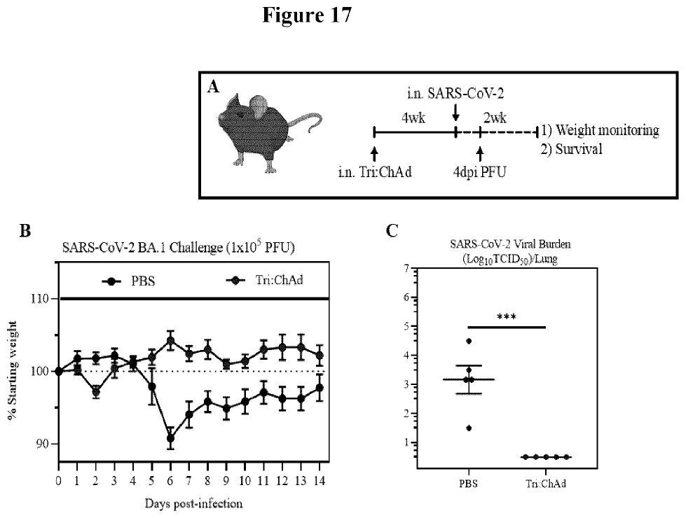

Weight loss was determined following i.n. infection with SARS-CoV-2 Omicron BA.1 in control (PBS) and i.n. Tri:ChAd-vaccinated animals (n=5 animals per group). As shown in FIG. 17B, the vaccinated animals exhibited insignificant weight loss as compared to unvaccinated control animals.

Lung viral titer enumeration 4 days post-infection with SARS-CoV-2 Omicron BA.1 in control (PBS) or i.n. Tri:ChAd-vaccinated animals was determined utilizing a VERO-E6 based assay and assessing for the presence of cytopathic effect (n=5 animals per group). As shown in FIG. 17C, little or no viral burden was detectable in the lung of vaccinated animals.

Thus, an adenoviral trivalent transgene such as Tri:ChAd is effective to protect against infection with an immune evasive SARS-CoV-2 variant (omicron variant).

### Example 12

The suitability of The AeroNeb® Solo nebulizing system for delivering adenoviral-vectored vaccine, via inhaled aerosol, to the lung for effective respiratory mucosal immunization in humans, was investigated.

Methods—The aerosol performance of the Aeroneb® Solo with AdHu5Ag85A was determined using standard procedures. Fill volume (FV), delivery time, emitted dose (ED) of vaccine aerosol available at the mouth collected on standard filters, and aerosol droplet size characteristics using the NGI Cascade Impactor operated at 15 Lpm were measured. The presence of vaccine in the NGI and on filters was determined by PCR. Salbutamol sulphate served as the tracer for saline droplets containing vaccine particles. Regional deposition of vaccine droplets in the lung was estimated from the particle size metrics of the carrier (salbutamol) aerosol. An indication of the available vaccine dose at the mouth was predicted from ED (emitted dose). The amount of aerosol-containing vaccine estimated to be deposited into the lung was calculated using ED in combination with particle size statistics. Viability of the aerosolized vaccine was determined by plaque forming assay.

Standard curves for salbutamol sulphate in PBS or 0.9N saline were determined on each experimental day. Samples were placed in matched quartz cuvettes and absorbances were read by UV/VIS Spectroscopy at λ=276 nm (Genesys 10S, Thermo Fisher Scientific, MA, USA). The time to completely aerosolize various fluid loads (0.2, 0.5, 1.0, 2.0 ml) of 0.9N saline was determined, operating the Aerogen Solo continuously on the bench with the Aerogen Control Unit. The Control Unit was turned off as soon as no visible aerosol was seen at the nebulizer exit and the time recorded. Three different Aerogen Solo nebulizers were each tested, measuring the time to dryness with the 4 fluid volumes. The volume selected was based on reproducibility of the measurement and, in addition, the time measured that was deemed manageable for subjects inhaling the vaccine in future clinical trials. The volume selected was used in all subsequent experiments.

Determination of Emitted Dose (ED)—Salbutamol Sulfate (SS) in PBS was used as a carrier aerosol to measure the amount of vaccine aerosol delivered to the mouth (ED). SS was mixed with AdHu5Ag85A vaccine (prepared at the Robert E.F. Vector Lab, McMaster Immunology Research Centre) and diluted in 0.9N sterile saline. A volume of 0.5 ml of this solution was loaded into the Aeroneb Solo Micropump nebulizer and aerosolized to dryness. Four different experimental setups were used to measure ED: i) running the nebulizer continuously with the Aerogen Control Unit. ii) collecting the drug on an absolute filter using a Copley Dosage Unit Sampling Apparatus (DUSA) (Copley Scientific, UK), operated at a constant flow rate of 15 L/min; iii) using the Copley Breath Simulator (BS1000, Copley Scientific, UK) with breath pattern settings at 15 bpm, I/E ratio 1:1, Vt=500 ml, then ‘breathing’ the Aerogen nebulizer to dryness and iv) In Vitro Ex Vivo in which 8 healthy human volunteers (4 males, 4 females) breathed salbutamol aerosol only from the Aerogen Solo and through an absolute filter placed at the mouth. Salbutamol Sulfate was recovered from either PALL or MSP filters for all methods and SS levels were detected by UV Spectroscopy at λ=276 nm. The time to aerosolize to dryness for each experimental run was also recorded.

Aerodynamic Particle Size Distribution (APSD)—APSD was measured with a Next Generation Impactor (NGI, MSP Corp, MN), operated at a constant, calibrated flow rate of 15 L/min (TSI Flowmeter 4000 Series). The effective cutoff diameters (ECDs) of the NGI stages at 15 Lpm ranged from 14.1-0.98 μm. The NGI standard inlet was used to interface the NGI with the Aerogen Solo nebulizer circuit. The nebulizer was operated via the Aerogen Control Unit, and aerosol collected in the NGI until dryness. The NGI was then dismantled and the drug in the cups processed according to laboratory SOP. Control sizings with salbutamol sulphate in saline were acquired; vaccine was then added to salbutamol sulphate and repeat sizings obtained to determine changes, if any, in yield and particle size distribution of aerosol. Salbutamol sulphate in saline was detected on the NGI stages by UV spectroscopy at λ=276 nm; qPCR analysis was used to determine the quantity of vaccine deposited in the cups of the NGI stages and on the end-stage filter. The following APSD statistics were determined: Mass Median Aerodynamic Diameter (MMAD), Geometric Standard Deviation (GSD) and the Respirable Fractions (RF—%<5.39 μm, RF—%<2.08-5.39 μm, %<2.08 μm) to estimate regional distribution in the lung.

The Respirable Dose (RD) of salbutamol deposited in the lung was estimated from the ED measurements and the Respirable Fractions obtained from the NGI APSD: RD=ED×% RF. These calculations are used to estimate the regional distribution of inhaled vaccine in the lung.

qPCR for quantification of adenovirus on NGI and filters—Test samples were provided in 2 mL of easyMag Lysis Buffer (bioMérieux, St. Laurant, QC) and stored at −80° C. until extraction. The samples were extracted by easyMag using the off-board lysis approach and eluted in a final volume of 50 uL. Five microliters or one-tenth of the extracted sample was then tested by a quantitative real-time Adenovirus PCR on the RotorGene Q (Qiagen, Toronto, ON) using the Qiagen QuantiTect Probe Kit and the primers and consensus probe. The assay targets a 103 bp conserved region of the adenovirus hexon gene and with 45 cycles of amplification, has a linear dynamic range between 5×10³ and 5×10⁸ copies/mL.

Viral Plaque Forming Assay to determine the viability of viral particles in aerosol droplets—293 cells were split from T150 flasks into 60 mm culture dishes to reach 80% confluence in 5 ml of MEM F-11 (10% FBS, 1% P/S, 1% HEPES) culture media on the day before viral plaque forming unit (PFU) assay. To carry out the PFU assay, 0.5 ml liquid vaccine was nebulized, successively 4 times (20, 20, 20 and 60 sec), and aerosol collected into sealed 50-ml Falcon tubes containing 10 ml of PBS++ (1×PBS with 0.01% CaCl₂ and 0.01% MgCl₂). Serial dilutions of collected aerosol viral samples were set up in 24 well plates containing PBS++ buffer. As a positive control, a sample was collected directly from the nebulizer reservoir before nebulization and diluted 10⁵, 5×10⁵ and 10⁶ times. Successive aerosol samples collected within 2 minutes of timeframe were diluted 10², 10³ and 10⁴ times, respectively. Culture medium was removed from 60 mm dishes of 293 cells and cells were then infected with 200 μl of serially diluted samples/dish. Each sample was set up in duplicate dishes. Cells were then incubated for 45 min in a 37° C. 5% CO₂ incubator. During incubation, MEMF11-agarose overlay was prepared by warming 2×MEMF11 media (10% FBS and 2% each of L-Glutamine, P/S and HEPES) in 44° C. and melting 1% agarose in a microwave oven, followed by cooling agarose to 44° C. in a water bath and mixing the equal volume of 1% agarose with MEMF11 media (10 ml/dish). Agar in dishes was allowed to solidify in room temperature inside a biological safety cabinet. The dishes were then incubated in a 37° C. 5% CO₂ incubator and the viral plaques were counted at days 5, 7 and 10.

Results—A FV (fill volume) of 0.5 ml was a reasonable volume to fully deliver the vaccine in saline. Subjects inhaled 0.5 ml of vaccine using tidal breathing in approximately 2 minutes. The ED of vaccine available at the mouth was approximately 50% of the loaded dose in the nebulizer. The fine particle fraction of the salbutamol aerosol with vaccine showed that 85% of the aerosol was contained in droplets <5.39 μm in diameter. Thus, the amount of aerosol available at the mouth and subsequently deposited in the lung was 42.5%. The estimated rate of viable vaccine from aerosol droplets generated by the nebulizer was 17.4%. Thus, our study indicates that the vaccine aerosol produced such can be efficiently deposited into human respiratory tract below the larynx.

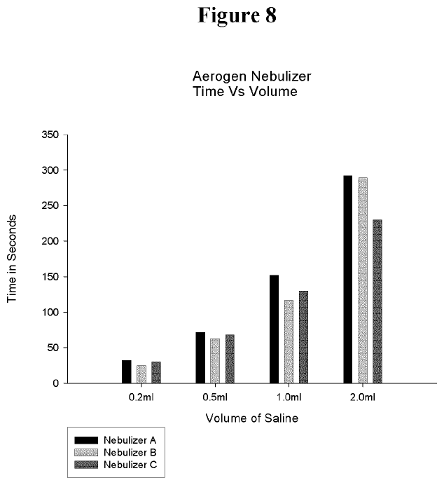

Optimal sample loading volume and nebulization time—Using the Aerogen Control Unit, we first determined the time for the Aerogen Solo to completely aerosolize fluid loads of 0.2-2.0 ml 0.9N saline and each volume was tested three times and in three nebulizers. Compared to other volumes, a Fill Volume (FV) of 0.50 ml delivered all the saline in the nebulizer reservoir in 67.8±3.7 sec (FIG. 8), which was deemed not an unreasonable amount of time for human subjects to remain comfortably breathing from the circuit. The time to completely aerosolize 0.50 ml was highly reproducible (CV=5.44%) (FIG. 8). A fill volume of 0.5 ml was thus selected in all subsequent in vitro measurements with the Aerogen Solo nebulizer.

Aerosol inhalation time for healthy humans—To determine aerosol inhalation time with human subjects, we first compared the nebulization time of 0.5 ml volume determined by using an Aerogen Control Unit with that by using Constant Flow of 151 lpm through a Dose Unit Sampling Apparatus (DUSA), or using a Breath Simulator with Vt of 500 ml, 15 bpm, linked to the Aerogen Solo nebulizer. The time to nebulize 0.5 ml saline using Constant Flow of 15 lpm through DUSA and Breath Simulator attached to the Aerogen delivery system was similar, being 71.5±7.12 secs and 70.5±2.60 secm, respectively (Table 2).

TABLE 2
Time (t) to nebulize 0.5 ml saline liquid under 4 operating
conditions using Aerogen Solo Micropump nebulizer

| | n | Time to nebulizer |
|---|---|---|
| Technique | (# of | 0.5 ml saline |
| Aerogen Solo nebulizer | runs) | mean ± sd-secs |
| 1. +Aerogen Control Unit giving continuous air | 3 | 67.7 ± 4.50 |
| flow through the nebulizer | | |
| 2. +Constant Air Flow of 15 lpm through the | 13 | 71.5 ± 7.12 |
| DUSA | | |
| 3. +Variable air flow using Breath Simulator | 14 | 70.5 ± 2.60 |
| with Vₜ = 500 ml, 15 bpm | | |
| 4. In Vitro/Ex-Vivo | 8 | 149.25 ± 8.0 |
| (Human Subjects, tidal breathing) | | |

Since the variance in human tidal breathing may influence the time required for completion of aerosol inhalation using Aerogen Solo nebulizer with 0.5 ml volume, we recruited a total of 8 healthy human subjects (4 males and 4 females) for determination of inhalation time. To this end, the inhalation time of completing aerosolized 0.5 ml saline was determined by using Aerogen Solo nebulizer and Control Unit plus filters and with human subject's nose clipped. The time to completely empty the Aerogen Solo using tidal breathing increased by approximately 70 secs to be 149.3±8.0 sec (Table 2) compared to operating the nebulizer under constant flow.

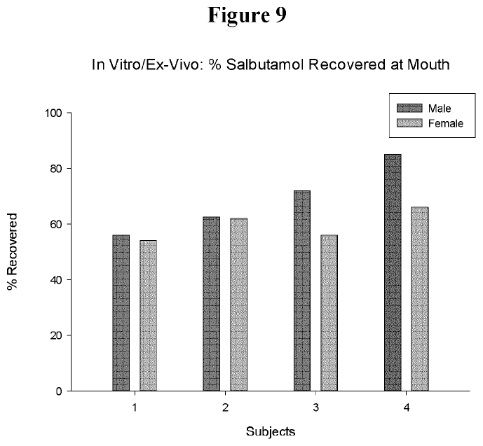

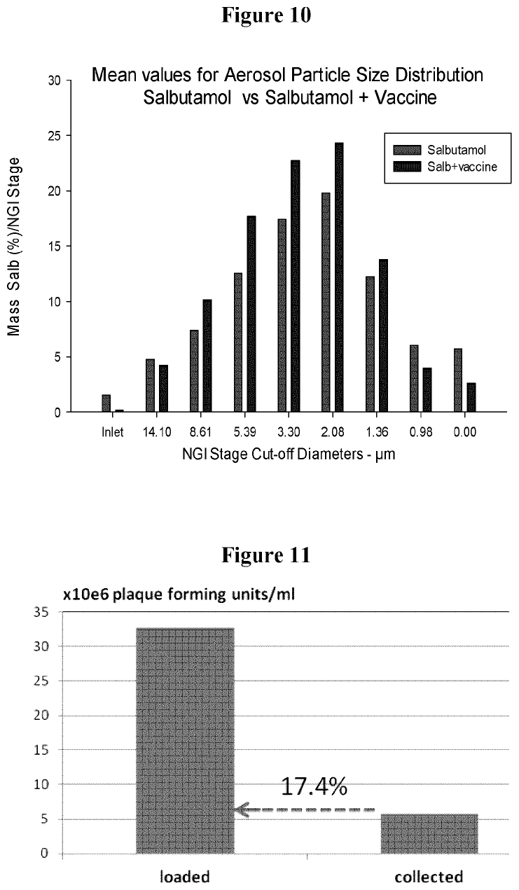

Emitted dose—Since expectedly there would be a loss of material during nebulization and within the system, it was important to determine the rate of the initially loaded vaccine available at the mouth. This was determined by using a tracer salbutamol sulphate in 0.5 ml loading buffer and with DUSA, Breath Simulator or human subjects described above, in the absence or presence of AdHu5Ag85A vaccine. In the absence of the vaccine, the mean amount of salbutamol recovered from the filter placed at the mouth was 41.8% and 44.7% by DUSA and Breath Simulator, respectively (Table 3A). This was on average 64% from 8 human subjects (Table 3A) and we did not observe a significant difference in the rates of emitted dose between males and females (FIG. 9). Addition of the vaccine on top of salbutamol slightly increased the emitted doses, being 54.50% and 48.8% with DUSA and Breath Simulator, respectively (Table 3B). Thus, we determined that the Emitted Dose of vaccine aerosol available at the mouth in humans was ˜50% of what was loaded into the nebulizer.

TABLE 3
Nebulization Time to ‘Empty’ and Emitted Dose
(ED) of Salbutamol Sulphate (3A) or Salbutamol Sulphate
with Vaccine (3B) from Aerogen Solo.
Table 3A: TIME to NEBULIZE & EMITTED DOSE of SALBUTAMOL
SULPHATE from AEROGEN SOLO - 625 μg in 0.5 ml

| | | Time to nebulize | ED - % Total |
|---|---|---|---|
| | | 0.5 ml SS | (625 μg) |
| Technique | n | (mean ± sd) - sec | mean ± sd |
| DUSA Constant | 5 | 74.0 ± 7.12 | 41.8 ± 8.57 ^{1,4} |
| Flow of 15 lpm | | | |
| Breath Simulator - | 8 | 74.3 ± 4.74 | 44.7 ± 7.57 ³ |
| Vₜ 500 ml, 15 bpm | | | |
| In Vitro/Ex-Vivo | 8 | 149.25 ± 8.0 | 64 ± 0.10 ⁵ |
| (Human Subjects) | | | |

Table 3B: TIME to NEBULIZE & EMITTED DOSE of SALBUTAMOL
SULPHATE with VACCINE from AEROGEN SOLO - 600 μg in 0.5 ml

| | | Time to nebulize | ED - % Total |
|---|---|---|---|
| | | 0.5 ml SS + Vaccine | (600 μg) |
| Technique | n | (mean ± sd) - sec | mean ± sd - % |
| DUSA - Constant | 4 | 78.5 ± 2.96 | 54.50 ± 2.29 ² |
| Flow of 15 lpm | | | |
| Breath Simulator - | 4 | 88.5 ± 2.96 | 48.8 ± 2.22 |
| Vₜ 500 ml, 15 bpm | | | |

¹ p = 0.551, 2-tailed P value, BS vs DUSA, no vaccine;
² p = 0.0142, 2-tailed P value, DUSA vs BS, with vaccine;
³ p = 0.396, 2-tailed P value, BS no vaccine vs with vaccine;
⁴ p = 0.0284, 2-tailed P value, DUSA no vaccine vs with vaccine;
⁵ p < 0.001, 2-tailed P value, BS vs IV/EV, no vaccine

Aerosol droplet size distribution—To determine the Aerodynamic Particle Size Distribution (APSD) of aerosol droplets, we used a Next Generation Impactor operated at a constant, calibrated flow rate of 15 L/min. We first measured the APSD of salbutamol sulphate aerosol generated from Aerogen Solo nebulizer as a control and then with the vaccine added to salbutamol sulphate in the nebulizer. Overall, the particle size statistics were the same for the salbutamol aerosols, with or without the vaccine added (p>0.099, Table 4) and we found the size characteristics of the aerosol produced by Aerogene Solo nebulizer to be well within the respirable range. A 85% Respirable Fraction (RF) of the aerosol was found to contain droplets less than 5.39 μm in size (Table 4; FIG. 10), ensuring a high deposition below the larynx. The remaining ˜15% of the aerosol was estimated to be deposited in the oropharynx (mouth and throat) (Table 4). Applying the (RF) %<5.39 μm to the Emitted Dose, the estimate of aerosolized vaccine available at the mouth and subsequently deposited in the lung was calculated to be 42.5% (0.85×0.50=0.425).

TABLE 4
Aerodynamic Particle Size Determination (APSD) Metrics
using the Next Generation Impactor (NGI).

| | Salbutamol | Salbutamol + Vaccine |
|---|---|---|
| | n = 5 | n = 4 |
| APSD Metrics from NGI | mean ± sd | mean ± sd |
| MMAD - μm | 3.25 ± 0.39 | 3.68 ± 0.20 |
| GSD | 3.31 ± 2.19 | 2.30 ± 0.08 |
| RF - % < 5.39 μm | 84.0 ± 4.03 | 85.2 ± 2.04 |
| RF - %2.08-5.39 μm | 69.1 ± 5.46 | 68.0 ± 1.99 |
| RF - % < 2.08 μm | 48.3 ± 5.81 | 48.4 ± 2.98 |

MMAD: Mass Median Aerodynamic Diameter; GSD: Geometric Standard Deviation.
NSD between APSD metrics (SS vs SS + V) when vaccine was added to Salbutamol Sulphate, p > 0.099

Viability of viral particles in aerosol—Since the Aerogen Solo Vibrating Mesh nebulizer used in our study operates on the design with a piezoelectric orifice plate which oscillates at high frequencies (22), the nebulization process could potentially reduce the viability of viral-vectored vaccine. We determined the viability of viral particles in aerosolized vaccine droplets collected into the buffer by using a viral plaque forming unit (PFU) assay and compared it with the sample within the nebulizer reservoir prior to nebulization. Before nebulization, on average there were a total of 32.75×10⁶ PFU present in the loaded liquid vaccine sample, compared to an average 5.7×10⁶ PFU from all collected aerosol samples (FIG. 11). This represented approximately an 18% viability of viral particles following nebulization.

Discussion—Aerosol immunization has several advantages over the parenteral route. Immunologically, it is able to induce long-lasting innate immune memory of airway macrophages for trained innate immunity against a broad range of respiratory pathogens besides its superior ability to elicit pathogen-specific tissue-resident adaptive immune responses (16, 23). Our study has fully characterized the use of The AeroNeb® Solo Micropump Nebulizer to produce aerosolized AdHu5-vectored vaccine for inhalational application in humans. We have established the vaccine fill volume, delivery time under tidal breathing, emitted dose to the mouth, aerosol particle sizes, and vaccine viability. Of these technical parameters are the findings that 42% of initially loaded vaccine is available to the mouth, 85% of the aerosol droplets are <5.39 μm in diameter, amenable to deposition into the major human respiratory airways below the larynx, and about 20% of aerosolized vaccine remains viable or infectious. Our data thus supports the suitability of The AeroNeb® Solo nebulizing system for delivering adenoviral-vectored vaccine, via inhaled aerosol, to the lung for effective respiratory mucosal immunization in humans.

When developing aerosol vaccine delivery strategies for human application, it is important to consider the choice of delivery device, fill volume and delivery time, emitted dose, particle sizes, and vaccine viability. Such considerations are critical to clinical aerosol vaccine development as they dictate the safety, vaccine loading dose, efficient delivery of bioactive vaccine to the respiratory tract, and consistency between vaccinees. However, these technical parameters may vary widely depending on the device, the nature and formulation of vaccine, and the targeted hosts. For these reasons, the information from clinical studies evaluating measles or MVA vaccine or from non-human primate studies testing Ad vaccines cannot be directly translated to human application with Ad vaccines. In this regard, our study represents the first where viral-based vaccine was delivered to the human respiratory tract according to the well-characterized technical parameters described above.

In summary, we have successfully defined a set of important technical parameters in using a single-patient use, commercially available nebulizer system to effectively deliver a recombinant AdHu5-vectored vaccine, via inhaled aerosol, to the human respiratory tract. This technology is currently being used in an ongoing phase 1b aerosol TB vaccine trial at McMaster University.

1. A trivalent transgene for use in a viral vaccine, wherein said trivalent transgene encodes:
i) a viral surface glycoprotein component comprising a signal peptide and a viral surface glycoprotein coupled to a transmembrane domain that functions to anchor the surface glycoprotein for extracellular expression;
ii) a viral nucleoprotein component that complexes or associates with viral RNA; and
iii) a viral RNA polymerase component.

2. The transgene of claim 1, wherein the transmembrane domain is a viral transmembrane domain.

3. The transgene of claim 1, wherein the transmembrane domain targets an extracellular vesicle.

4. The transgene of claim 1, wherein the transmembrane domain is from a protein selected from the group consisting of CD63, CD9, CD81, CD82, LAMP2B, CdaA, VSVG, Junin virus glycoprotein, Lassa fever virus glycoprotein, LCMV glycoprotein, SARS-CoV-2 glycoprotein, Tamiami virus glycoprotein, Guanarito virus glycoprotein, Machupo virus glycoprotein, Sabia virus glycoprotein and Parana virus glycoprotein.

5. The transgene of claim 1, wherein the surface glycoprotein component is derived from a first virus and the transmembrane domain is derived from a second virus.

6. The transgene of claim 1, wherein the RNA polymerase component comprises viral RNA-dependent RNA polymerase (RdRp) or a conserved region thereof.

7. The transgene of claim 1, wherein the surface glycoprotein component is linked to the nucleoprotein and RNA polymerase components with a self cleaving peptide.

8. The transgene of claim 7, wherein the peptide is a 2A peptide.

9. The transgene of claim 1, wherein the surface glycoprotein component, the nucleoprotein component and the RNA polymerase component is derived from a positive single-stranded RNA virus.

10. The transgene of claim 9, wherein the positive single-stranded RNA virus is a coronavirus.

11. The transgene of any one of claim 9 or 10, wherein the surface glycoprotein component comprises a spike protein or an immunogenic fragment thereof.

12. The transgene of claim 11, wherein the surface glycoprotein component comprises a receptor binding domain fragment of the spike protein.

13. The transgene of any one of claims 9-12, wherein the nucleoprotein component comprises a coronaviral nucleoprotein or immunogenic fragment thereof.

14. A vaccine comprising a trivalent transgene as defined in any one of claims 1-13.

15. The vaccine of claim 14, which is a DNA vaccine, an mRNA vaccine or a viral-vectored vaccine.

16. The vaccine of claim 14, which is a DNA viral vector vaccine comprising a viral vector from a poxvirus, adenovirus, adeno-associated virus, herpes simplex virus or cytomegalovirus.

17. The vaccine of claim 14, which is a RNA viral vector vaccine comprising a viral vector from a vesicular stomatitis virus, retrovirus, lentivirus, Sendai virus, measles-derived vaccine, Newcastle disease virus, alphavirus or a flavivirus.

18. A replication-incompetent E1/E3-deleted adenoviral vector.

19. The replication-incompetent adenoviral vector as defined in claim 18, comprising a transgene, wherein the transgene is incorporated within the E1/E3 deletion.

20. The replication-incompetent adenoviral vector as defined in claim 19, wherein the transgene is a trivalent transgene as defined in any one of claims 1-13.

21. The replication-incompetent adenoviral vector as defined in any one of claims 18-20, wherein the vector comprises the sequence of SEQ ID NO: 7.

22. A method of vaccinating a mammal against a viral infection comprising treating the mammal with a vaccine as defined in any one of claims 14-17 or a vector as defined in any one of claims 19-21.

23. A kit comprising an adenovirally-vectored vaccine in combination with a nebulizer.

24. A kit comprising a vaccine as defined in any one of claims 14-17 or a vector as defined in any one of claims 19-21 in combination with a nebulizer.

25. A method of vaccinating a mammal against infection by a virus comprising administering to the mammal an adenovirally-vectored vaccine with a nebulizer.

26. The method of claim 25, wherein the vaccine comprises a transgene as defined in any one of claims 1-13.

27. A trivalent transgene for use in a vaccine effective against a positive single-stranded RNA virus, wherein said trivalent transgene encodes:
i) a first viral surface glycoprotein component comprising a signal peptide and a viral surface glycoprotein coupled to a transmembrane domain that targets an extracellular vesicle and functions to anchor the surface glycoprotein to the vesicle for extracellular expression; and
ii) a second protein comprising a viral nucleoprotein component that complexes or associates with viral RNA and a viral RNA polymerase component,
wherein the first glycoprotein component and the second protein are linked with a self-cleaving peptide.

28. The transgene of claim 27, wherein the RNA virus is a coronavirus.

29. The transgene of claim 28, wherein the coronavirus is a SARS-CoV2.

30. The transgene of claim 29, wherein the surface glycoprotein component is the receptor binding domain of the spike glycoprotein and the transmembrane domain is a heterologous viral transmembrane domain.

31. A vaccine comprising a trivalent transgene as defined in any one of claims 27-30.

32. The vaccine of claim 31, which is a DNA vaccine, an mRNA vaccine or a viral-vectored vaccine.

33. The vaccine of claim 32, which is a DNA viral vector vaccine.

34. The vaccine of claim 33, which is an adenoviral vaccine.

35. A method of vaccinating a mammal against a viral infection comprising treating the mammal with a vaccine as defined in any one of claims 31-34.
